(19) United States
(12) Patent Application Publication
(10) Pub. No.: US 2006/0015264 A1
(43) Pub. Date: Jan. 19, 2006

(54) Interfering stem-loop sequences and method for identifying

(76) Inventors: Andrew McShea, Kingston, WA (US); Andrew M. Antoniewicz, Mukilteo, WA (US)

Correspondence Address: COMBIMATRIX CORPORATION, 6500 HARBOUR HEIGHTS PARKWAY, MUKILTEO, WA, 98275, US

(21) Appl. No.: 10/860,095

(22) Filed: Jun. 2, 2004

(51) Int. Cl. G01N 33/48; G01N 33/50; G06F 19/00

(52) U.S. Cl. 702/20

(57) ABSTRACT

A method for identifying stem-loop structures within a genome is provided. A plurality of stem-loop structures, compounds of stem-loop structures, pharmaceutical compositions of stem-loop structures, and treatment methods for affecting a condition or disease in an organism using stem-loop structures is provided. The method is for rapidly identifying and screening small inhibitory stem-loop structures of RNA or DNA sequences of any genome, wherein those sequences or combinations thereof can be administered to obtain a desirable biological affect in a human or other organism for treatment of a condition or a disease. The method is used for rapidly identifying and screening small inhibitory stem-loop structures of a viral RNA (viRNA), wherein the viRNA's prevents death in transfected cells programmed for cell death thus providing siRNA-type compositions for use in treating inflammatory conditions in humans or other species.

### TECHNICAL FIELD OF THE INVENTION

The present invention provides a method for identifying stem-loop structures within a genome, a plurality of different stem-loop structures, compounds of stem-loop structures, pharmaceutical compositions of stem-loop structures, and treatment methods for affecting a condition or disease in an organism using stem-loop structures. Specifically, the present invention provides a method for rapidly identifying and screening small inhibitory stem-loop structures of RNA or DNA sequences of any genome, wherein those sequences or combinations thereof can be administered to obtain a desirable biological affect in a human or other organism for treatment of a condition or a disease. More specifically, the present invention provides a method for rapidly identifying and screening small inhibitory stem-loop structures of a viral RNA (viRNA), wherein the viRNA's prevents death in transfected cells programmed for cell death thus providing compositions for use in treating inflammatory conditions in humans or other species.

### BACKGROUND OF THE INVENTION

Viral pathogens, posing a physiological threat to healthy subjects, utilize multiple mechanisms to evade attack from the host immune system. Representative articles that teach viral mechanisms to evade attack include the following: Viral mimicry of cytokines, chemokines and their receptors, Alcami A. Nat Rev Immunol. 2003 January 3(1):36-50; To kill or be killed: viral evasion of apoptosis, Benedict C A, Norris P S, Ware C F. Nat Immunol. 2002 November 3(11):1013-8; Viral exploitation and subversion of the immune system through chemokine mimicry, Murphy P M, Nat Immunol. 2001 February 2(2):116-22; Poxviral mimicry of complement and chemokine system components: what's the end game?, Kotwal G J. Immunol Today, 2000 May 21(5):242-8. The disclosure of the aforementioned articles is incorporated by reference herein. Viral evasion mechanisms can be directly linked to the expression of viral gene products within virally infected cells, and presumably, such evasion mechanisms have evolved to protect virally infected cells from recognition by the host immune system. Most viral species have gene products, such as proteins, that have been shown to have a role in immune evasion.

Some of these virally produced proteins have considerable amino acid sequence homology with the host cell proteins that are involved in immune response regulation (e.g. cytokines), programmed cell death, or antigen presentation. Other viral gene proteins have no obvious amino acid sequence homology to host proteins but have potent immunomodulatory activity. A number of viral proteins have been shown to be critical to viral pathogenicity, and deletion of these the genes that cause expression of such proteins can attenuate the virus pathogenicity. Current research on direct biological activities of RNA in mammalian cells, plants, worms, and fruit flies has demonstrated that certain types of RNA transcripts can directly regulate expression of other genes through a mechanism called RNA interference, wherein such RNA's interfere with cellular function and mechanisms by selective binding with cellular RNA that is complementary to the interfering RNA. Representative articles that teach RNA interference include the following: Nucleic Acid-Based Immune System: the Antivial Potential of Mammalian RNA Silencing, Gitlin, L., and Andino, R., J. of Virology, July 2003, p. 7159; Computational identification of Drosophila microRNA genes, Lai, E. C., Tomancak, P., Williams, R. W., and Rubin, G. M., Genome Biology, 2003, 4:R42; Identification of Drosophila MicroRNA Targets, Stark, A., Brennecke, J., Russell, R. B., and Cohen, S. M., PloS Biology, Vol. 1, Issue 3, p. 001. These RNA transcript studies indicated that short double-stranded segments of synthetic RNA could be used to inhibit expression of a specific protein when the synthetic RNA is complementary in base sequence to the RNA transcript of the specific protein. Such small interfering RNA's are termed siRNA's. Such siRNA's have become excellent tools for the inhibition of gene expression.

Additionally, current research has also shown that siRNA-like effector molecules, called miRNA's, may also exist in a variety of organisms, including homo sapiens. Representative articles that teach miRNA and RNA interference in mammals include the following: Bartel, D. P., MicroRNAs: genomics, biogenesis, mechanism, and function, Cell 2004, Jan. 23, 116(2):281-97; McManus, M. T., Sharp, P. A., Gene silencing in mammals by small interfering RNAs, Nat Rev Genet. 2002 October; 3(10):737-47. These endogenous miRNA molecules have also been shown to inhibit the expression of specific RNA transcripts and may form part of a regulatory network that regulates the phenotype of a cell without directly encoding a protein product but instead by selective binding to complementary RNA's. The widespread occurrence of miRNA's suggests that these molecules have played a significant role in the evolutionary success of a diverse group of organisms, including homo sapiens.

Viruses are also subject to regulation by synthetic inhibitory RNA's. Viruses share the transcriptional machinery of the host cell with the host cell, and research has demonstrated that viral replication, in vitro and in vivo, can be effectively inhibited using siRNA's targeted against specific viral genes, wherein the siRNA is complementary to the viral genes to allow base-to-base binding. A representative article that teaches siRNA targeting of specific viral genes includes the following: McCaffrey A P, Nakai H, Pandey K, Huang Z, Salazar F H, Xu H, Wieland S F, Marion P L, Kay M A., Inhibition of Hepatitis B virus in mice by RNA interference, Nat. Biotechnol. 2003 June; 21(6):639-44. What role endogenous miRNA's have on viral replication is unclear; however, given the widespread occurrence of miRNA's, the evolved and efficient nature of the viral genome, the high frequency of miRNA-like stem-loop structures in viral genomes, and the propensity of certain RNA-type viruses to recombine with host cell genetic material, viral genomes may possibly encode their own miRNA sequences, which would provide viruses with the ability to regulate expression as well as other functions.

Viral pathogenesis may be attributable to endogenous miRNA sequences in the viral genome, rather than, or in addition to, viral genes that encode for proteins that are deleterious to the host. Identification of miRNA sequences in a virus, hereinafter referred to as viRNA, may also provide information about the host cell pathway that is targeted by a virus and thus provide a better understanding of viral pathogenesis. If multiple viRNA's affect the same host cell transcript, then such a result might also suggest a pan-tropic approach for anti-viral therapies or for the treatment of other conditions or diseases. In either case, the identification of a conserved viRNA motif that is required for viral replication may be used to develop a therapeutic strategy for treatment of a number of conditions or diseases. Similarly, identification of interfering RNA's or DNA's in any conserved genetic motif may be used to develop a therapeutic strategy for treatment of a number of conditions or diseases.

Previous research on interfering RNA's has not investigated the existence of nor the biological activity of viRNA's with respect to how such RNA's may thwart host cell defense mechanisms. Instead, previous research has focused on the role of host cell interfering RNA's that target viruses. There is a great deal of interest in identifying host cell miRNA's that inhibit viral replication. Indeed, there is a great deal of interest in identifying interfering RNA's (RNAi) to treat a variety of diseases, and many such molecules have some biological activity. However, the search for the best RNAi to modify biological function (i.e., the most biologically potent) may be improved by screening organisms such as viruses and other “cellular parasites,” which have to affect this function on a routine basis. Representative articles that teach modifying biological function by use of interfering RNA's include the following: Wiebusch L, Truss M, Hagemeier C., Inhibition of human cytomegalovirus replication by small interfering RNAs, J Gen Virol., 2004 January; 85(Pt 1): 179-84; Davidson, B. L., Hepatic diseases—hitting the target with inhibitory RNAs, N Engl J Med., 2003 Dec. 11, 349(24):2357-9.He M L, Zheng, B., Peng, Y., Peiris, J. S., Poon, L. L., Yuen, K. Y., Lin, M. C., Kung, H. F., Guan Y., Inhibition of SARS-associated coronavirus infection and replication by RNA interference, JAMA, 2003 Nov. 26, 290(20):2665-6; Butz, K., Ristriani, T., Hengstermann, A., Denk, C., Scheffner, M., Hoppe-Seyler, F., siRNA targeting of the viral E6 oncogene efficiently kills human papillomavirus-positive cancer cells, Oncogene, 2003 Sep. 4, 22(38):5938-45; Wang Q C, Nie Q H, Feng Z H, RNA interference: antiviral weapon and beyond, World J Gastroenterol., 2003 August 9(8):1657-61; Chang J, Taylor J M, Susceptibility of human hepatitis delta virus RNAs to small interfering RNA action, J Virol. 2003 September, 77(17):9728-31; Andino, R., RNAi puts a lid on virus replication, Nat Biotechnol. 2003 June, 21(6):629-30; McCaffrey, A. P., Nakai, H., Pandey, K., Huang, Z., Salazar, F. H., Xu, H., Wieland, S. F., Marion, P. L., Kay, M. A., Inhibition of hepatitis B virus in mice by RNA interference, Nat Biotechnol. 2003 June 21(6):639-44; Wilson, J. A., Jayasena, S., Khvorova, A., Sabatinos, S., Rodrigue-Gervais, I. G., Arya, S., Sarangi, F., Harris-Brandts, M., Beaulieu, S., Richardson, C. D., RNA interference blocks gene expression and RNA synthesis from hepatitis C replicons propagated in human liver cells, Proc Natl Acad Sci USA, 2003 Mar. 4, 100(5):2783-8; Ge Q, McManus, M. T., Nguyen, T., Shen, C. H., Sharp, P. A., Eisen, H. N., Chen, J., RNA interference of influenza virus production by directly targeting mRNA for degradation and indirectly inhibiting all viral RNA transcription, Proc Natl Acad Sci USA, 2003 Mar. 4, 100(5):2718-23; Jia, Q., Sun, R., Inhibition of gammaherpesvirus replication by RNA interference, J. Virol. 2003 March, 77(5):3301-6; Kapadia, S. B., Brideau-Andersen, A., Chisari, F. V., Interference of hepatitis C virus RNA replication by short interfering RNAs, Proc Natl Acad Sci USA 2003 Feb. 18, 100(4):2014-8; Pooggin, M., Shivaprasad, P. V., Veluthambi, K., Hohn, T., RNAi targeting of DNA virus in plants, Nat Biotechnol. 2003 February, 21(2):131-2.

Viral genomes are thought to represent some of the most evolved genomic architecture in nature, due to their highly compact and efficient use of nucleic acids, short doubling time, large numbers of mutated progeny, and high selective pressure. Thus, when trying to identify the most biologically potent inhibitory RNA motif's, focusing upon viRNA rather than miRNA's of a host cell may provide a better approach, wherein more biologically potent inhibitory RNA motifs may be identified for use in treatments. Such viRNA motifs may specifically inhibit the transcripts from a single gene or may represent multifunctional RNA motifs that inhibit multiple genes with the same viRNA motif.

The phenomenon of post-transcriptional gene silencing (PTGS), or inhibition of mRNA translation by homologous double stranded RNA (dsRNA) offers a powerful tool for understanding the functional significance of individual genes. siRNA molecules can be used as highly selective probes to screen loss of function phenotypes in human cell-based assays and, thereby, identify genes critical to the expression of a specific phenotype. Bioinformatics is key to developing libraries of siRNA molecules for selective gene silencing. However, bioinformatics can also be used to identify gene sequences in pathogenic viruses that may encode for RNA moieties, which then modulate human host cell functions.

Novel therapeutics with anti-inflammatory or immune modulatory activity used to treat a variety of ailments that are very significant problems for human health. These include autoimmune and inflammatory diseases, such as arthritis, lupus, and type I diabetes and also complications of other conditions where the human immune system needs to be ‘reigned-in’ such as organ transplantation and sepsis.

One of the critical issues in developing new drugs is that, although many of the gene products have been identified by name and sequence, research has proven to be challenging when attempting to identify which gene products define exactly which pathways. Given the high level of redundancy in biological systems, research can be challenging when attempting to determine where novel points of intervention in a cellular pathway are located in terms of identifying a target or receptor for a drug.

High throughput approaches to ascribing functional significance to genes, usually described as “functional genomics” have become much more powerful with the advent of inhibitory RNA technologies. Representative articles that teach functional genomics include the following: Elbashir, S., Martinez, J., Patkaniowska, A., Lendeckel, W. and Tuschl, T. Functional anatomy of siRNAs for mediating efficient RNAi in Drosophila melanogaster embryo lysate, EMBO J., 20, 6877-6888 (2001); Harborth, J., Elbashir, S. M., Bechert, K., Tuschl, T. and Weber, K., Identification of essential genes in cultured mammalian cells using small interfering RNAs, J. Cell Sci., 114, 4557-4565 (2001); Lewis, D. L., Hagstrom, J. E., Loomis, A. G., Wolff, J. A. and Herweijer, H. Efficient delivery of siRNA for inhibition of gene expression in postnatal mice. Nature Genet., 32, 107-108 (2002); DiTullio, R. A., Jr, Mochan, T. A., Venere, M., Bartkova, J., Sehested, M., Bartek, J. and Halazonetis, T. D., 53BP1 functions in an ATM-dependent checkpoint pathway that is constitutively activated in human cancer, Nature Cell Biol., 12, 998-1002 (2002); Hasuwa, H., Kaseda, K., Einarsdottir, T. and Okabe, M. Short 5′-phosphorylated double-stranded RNAs induce RNA interference in Drosophila, FEBS Lett., 532, 227-230 (2002). Inhibitory RNA screening approaches are being used to screen loss of function phenotypes in cell-based assays to try to identify the most critical elements of the cellular machinery responsible for a wide range of phenotypes, including those pathways involved in the apoptosis and signaling cascades. Representative articles that teach screening approaches include the following: Shirane, D., Sugao, K., Namiki, S., Tanabe, M., Iino, M., Hirose, K., Enzymatic production of RNAi libraries from cDNAs, Nat Genet. 2004, February, 36(2):190-6; Kumar, R., Conklin, D. S., Mittal, V., High-throughput selection of effective RNAi probes for gene silencing, Genome Res. 2003, October, 13(10):2333-40; Aza-Blanc, P., Cooper, C. L., Wagner, K., Batalov, S., Deveraux, Q. L., Cooke, M. P., Identification of modulators of TRAIL-induced apoptosis via RNAi-based phenotypic screening, Mol Cell. 2003, September, 12(3):627-37; Silverstein, A. M., Mumby, M. C., Analysis of protein phosphatase function in Drosophila cells using RNA interference, Methods Enzymol. 2003, 366:361-72.

Because of the efficacy of siRNA in mammalian cell culture systems, the loss of function experiments can be carried out in fairly elaborate in vitro models. Construction of large siRNA libraries for screening can be very expensive, and there is no guarantee that the siRNA molecules will act in an entirely specific manner. A representative article that teaches siRNA library construction includes the following: Jackson, A. L., Bartz, S. R., Schelter, J., Kobayashi, S. V., Burchard, J., Mao, M., Li, B., Cavet, G., Linsley, P. S., Expression profiling reveals off-target gene regulation by RNAi, Nat Biotechnol. 2003 June, 21(6):635-7. An alternative approach is to look at naturally occurring miRNA's. Representative articles that teach miRNA's includes the following: Bartel, D. P., MicroRNAs: genomics biogenesis, mechanism, and function, Cell 2004 23, 116(2):281-97; Lee, Y., Jeon, K., Lee, J. T., Kim, S., Kim, V. N., MicroRNA maturation: stepwise processing and subcellular localization, EMBO J. 2002 Sep. 2, 21(17); Doench, J. G., Petersen, C. P., Sharp, P. A., siRNAs can function as miRNAs, Genes Dev. 2003 Feb. 15, 17(4): 438-442; Zeng, Y., Yi, R., Cullen, B. R., MicroRNAs and small interfering RNAs can inhibit mRNA expression by similar mechanisms, Proc Natl Acad Sci USA, 2003, Aug. 19, 100(17): 9779-9784; Stark, A., Brennecke, J., Russell, R. B., Cohen, S. M., Identification of Drosophila MicroRNA Targets, PLoS Biol. 2003, December, 1(3).

miRNA's, which are naturally occurring, short, stem-loop structures of endogenous inhibitory RNA, have been identified in a variety of organisms and through a mechanism of RNA processing, acquire siRNA-like RNA interference activity. Studying miRNA-like molecules may provide important insight into which elements of the cellular machinery are most critical for cellular function. Indeed such an approach may show that miRNA-like molecules affect their phenotype through the inhibition of multiple targets. The key advantage with using a more empirical approach to screening siRNA's from nature, rather than trying to design a specific siRNA for every gene in the genome, is that such an approach may determine, at least in part, which transcripts are inhibited by the siRNA based on the homology of the nucleotide sequence and one of the siRNA strands.

miRNA's have been widely described and are thought to serve part of an autoregulatory process that is used to control transcription. Research has focused on plant and animal viruses, which have their own unique genomes but utilize the host transcriptional machinery. In addition to the viral genome encoding proteins to evade host immuno surveillance, viral genomes may also encode inhibitory RNA molecules that may modulate the immune and inflammatory responses of the host organism.

The potential of siRNA approaches to human disease therapy have received a great deal of attention. Identification and validation of biologically efficacious siRNA molecules has commercial value either as a possible therapeutic or as a tool that identifies novel druggable targets for small molecule approaches. Whilst many delivery challenges remain in the direct application of nucleic acid based therapies in humans, the efficacy of siRNA as a development tool for target discovery in vitro is significant. There are multiple approaches to the identification of novel biological targets that are candidates for modulation by siRNA or subsequently by small molecules.

One approach is to generate a large library of siRNA molecules that have been designed against a subset of all known genes in the human genome. Using standard high throughput approaches and in vitro assays, such an approach can be used to screen each of these molecules for a biologically relevant phenotype. There are numerous problems with this approach that include the following: (1) making the siRNA is expensive and once made, the siRNA is difficult to characterize with respect to what siRNA was actually made; (2) the rules for designing siRNA molecules are not sufficiently clear to allow production of inhibitory RNA's with relevant specificity profiles; (3) the target genes chosen for inhibition by an siRNA may not be the correct target; and (4) a single gene, single phenotype approach may not be a meaningful or may not even be attainable as a high throughput approach to a therapy.

An alternative approach is to screen inhibitory RNA molecules that have been identified in nature. Inhibitory RNA's, termed microRNA's (miRNA), have been identified as stem-loop structures in a wide variety of organisms and have been shown to be processed to produce a double stranded RNA molecule that has similar activity to synthetically made siRNA molecules. An empirical approach is to experimentally screen viral genomes as a source of inhibitory RNA molecules to identify biologically significant targets for therapeutics development. Viral genomes are chosen because they are likely to possess the most evolved/selected inhibitory RNA motifs, and to this date, numerous coding (i.e., translated) regions of viral genomes have been demonstrated to modulate the immune-surveillance, immune response and inflammatory response of the host. Since inhibitory RNA structures act to inhibit a homologous or complimentary transcript, depending on which strand of an RNAi molecule one is referring to, the target of gene silencing can be identified based on the nucleotide sequence of the stem loop structure. Thus research may identify the relationship between a phenotype of a viRNA molecule in a biological assay with the genotype of the genes that have been targeting for silencing. However, the relationship between viRNA and single gene targets may be more complex due to “off-target” effects. Jackson, A. L., et al., Nat. Biotechnol., June, 21(6):635-7, 2003.

An advantage of a more empirical approach to screening siRNA's is that the target(s) of inhibition can be identified based on the homology of one of the siRNA strands. Thus biological phenotype can be related to genotype. A completely random approach to generating siRNA libraries may also be viable; however, the large number of potential siRNA's that could be made randomly (4×10E17 to 4×10E23) make it difficulty to screen in most biological assays. In addition, this sort of library is unlikely to be constructed without considerable sequence bias. Therefore, by pre-selecting those inhibitory RNA candidates based on sequences found in viruses, there would be a reduction in the number of RNAi molecules that need to be screened to identify useful phenotypes in vitro.

An empirical approach provides a means to obtain useful information on the patho-physiological mechanisms of viral disease. Identifying and verifying the biological activity of non-translated nucleic acid sequences based on a screening approach may be very helpful for understanding the pathology of human infectious diseases. In addition, this approach identifies possible biological function of a component of a virus without requiring the use of “live virus,” which has significant advantages for developing and understanding the etiology of a viral infection that can be difficult to model in a laboratory setting and for preventing a serious biological safety threat. Development of a predictive algorithm that can identify biologically active inhibitory RNA structures in viral genomes may also provide novel targets for viral therapy. Additionally, Such an approach may yield useful information on key cellular processes that can be inhibited for pharmacological reasons. Both the targets and the viRNA molecules that are identified in a bioinformatic and functional screen will enhance the ability to develop a portfolio of targets for screening and subsequent steps in the lead discovery process.

An empirical approach requires a computational method to easily identify, categorize and rank stem loop structures in a nucleic acid sequence. A previous study focused on the construction of computational methods for efficiently designing oligonucleotide probes for hybridization experiments. This study culminated in an automated system that designs optimized probes for hybridization experiments based on large lists of accession numbers. This system has been successfully deployed as part of the commercially available Combimatrix ‘CustomArray™” microarray product. Using pre-existing segments of the retrieval code, the study developed a system for rapidly retrieving nucleic acid sequences for screening based on accession number. A computer program for identifying RNA stem-loop structures was required to permit screening of stem-loop motifs in a high throughput fashion.

Computer programs for identifying RNA or DNA folded structures are focused on finding a thermodynamically optimal structure for a sequence of interest. One such computer program is mFold. Zuker, M., Science 244, 48-52 (1989); Jaeger, J. A. Turner, D. H., and Zuker, M. Proc. Natl. Acad. Sci. USA, 86, 7706-7710 (1989). The computer program mFold attempts to predict a low free energy RNA secondary structure or folding for a given RNA or DNA base sequence. These predicted RNA structures are subsequently examined to determine their function and how secondary and tertiary structures interact with various cellular machinery. The mFold program utilizes a large number of thermodynamic parameters to model the predicted free energy of a specific folding and utilizes several algorithms, including dynamic programming, to find these optimal structures. mFold, however, tries to find the low energy folding for an entire sequence and not a portion of a sequence. Depending on the structure of the sequence, a potential miRNA site may not appear as part of the final folded structures. Even if a potential miRNA candidate appears in the mFold results, there is also an additional time cost to examine the folding results and pick any miRNA candidates from the resulting folded structures. In addition, many of the sequences of interest are large (100k bases or larger). These sequences will take a prohibitively long time to calculate their optimal structure because of the order N³ calculation required for mFold's algorithm to complete its prediction. Therefore, mFold will not locate potential stem-loop structures along an entire gene sequence that are characteristic of interfering RNA's and will not produce the sort of output that could be used to easily compare and rank the quality of putative stem-loop structures.

Another computer program related method for identifying RNA or DNA folded structures is miRseeker. A three-part computational pipeline is used in the miRseeker method. The method begins searching for potential miRNA candidates in highly conserved regions of related species. Conservation is determined by using the gene global alignment tool AVID to align two genomes. From the identified conserved regions, the following are eliminated from consideration: exons, transposable elements, snRNA, snoRNA, tRNA, and rRNA genes. Of the remaining genes, locations of miRNA genes, called regions, are identified in 100 unit segments, where a segment is a base pair or a base with a gap, and there are no more than 13% gaps or 15% mismatches. If a section of the sequence is identified as being conserved, a small area surrounding this conserved region is extracted and the mFold version 3.1 software is used to calculate a set of potential foldings for this extracted region. An overlap of 10 bases from each region is used because folding programs do not necessarily identify characteristic pre-miRNA structures if folded within the context of longer RNA's because of base-pairing with non-miRNA sequences. The results of the mFold are then examined and any foldings with “arm” structures are kept for downstream analysis. The miRseeker method requires that there be two related species for this examination. Also it requires that any miRNA candidate be present only inside these conserved regions. The mFold software is used to fold the selected conserved area; therefore, the structures found will be thermodynamically optimum structures, which does not identify all possible base pairings that could be found as a stem-loop structure.

Due to the inherent limitations of current bioinformatics approaches in the art to finding potential stem-loop structures, there is a need for a computer program and method that can be used to quickly and efficiently identify all potential stem-loop structures by scanning an entire genome of any size without restrictions based on matching gene sequences of two genomes or based on finding only thermodynamically optimum structures of a gene sequence or a portion of a gene sequence. Once a method for identifying potential stem-loop structures is obtained, there is a need for a method to efficiently screen such structures for useful biological activity in the treatment of a disease or condition.

### SUMMARY OF THE INVENTION

The present invention provides a method for efficiently identifying and screening a genome for stem-loop structures from which inhibitory RNA or DNA base sequences may reside. Additional, the present invention provides a plurality of different stem-loop structures, compounds of stem-loop structures, and pharmaceutical compositions of stem-loop structures. The present invention further provides treatment methods using stem-loop structures for affecting a condition or disease in an organism. Specifically, the present invention provides a method for rapidly identifying and screening small inhibitory stem-loop structures of RNA or DNA sequences of any genome, wherein those sequences or combinations thereof can be administered to obtain a desirable biological affect in a human or other organism for treatment of a disease or condition. The stem portion of a stem-loop structure is compared to a genome of a target organism to find complementary structures, wherein stems that match to a portion of the target genome are further screened for biological activity in a target cell. More specifically, the present invention provides a method for rapidly identifying and screening small inhibitory stem-loop structures of a viral RNA (viRNA), wherein the viRNA's prevent death in transfected cells programmed for cell death thus providing compositions for use in treating inflammatory conditions in humans.

The present invention provides a method on a computer for identifying stem-loop structures that are on a candidate genome, wherein those structures can be useful for treatment of a condition or disease in a target organism. The method, preferably using a computer data processing system, comprises reading a base sequence of the candidate genome from a computer readable medium, locating a window of the base sequence, and finding an optimum base sequence pairing by calculation of a stem-loop structure quality using a dynamic programming method to optimally fold the window to maximize matching of base pairs inside the window.

Alternatively, the computer method for screening comprises reading a base sequence of the candidate genome from a computer readable medium, pairing bases in a window of the base sequence by matching the bases in a first half of the window with the bases in a second half of the window, and forming a folded paired base window. The method then searches the folded paired base window for a consecutively bound base pair grouping, and finds an optimum base sequence pairing that allows calculation of a stem-loop structure quality. An optimum pairing is obtained by calculating a loop quality in a loop region of the consecutively bound base pair grouping using an loop-end dynamic programming method for matching the bases in the loop region extending away from the consecutively bound base pair grouping and calculating a open quality in an open region of the consecutively bound base pair grouping using an open-end dynamic programming method for matching the bases in the right region extending away from the consecutively bound base pair grouping.

The present invention provides a matching method for identifying high-scoring candidate stem-loop structures on a candidate genome by screening the stem-loop structures identified using either version of the aforementioned computer program by ranking the stem-loop structures according to stem-loop structure quality, heterogeneity, and conservation to form a subset of high-ranking stem-loop structures. Candidate stem-loop structures are selected from the subset of high-ranking stem-loop structures by comparing the base sequence of each structure from the subset of high-ranking stem-loop structures to the base sequence of the target organism by using a BLAST method. High-scoring candidate stem-loop structures are selected from the candidate stem-loop structures by using a parsing method. The high-scoring candidate stem-loop structures are those structures that have significant base matches to the target genome.

The present invention provides a screening method that screens the high-scoring candidate stem-loop structures for identifying interfering RNA drug candidates by synthesizing the high-scoring candidate stem-loop structures using a phosphoramidite chemistry method. The synthesized structures are transfected into cells taken from a target organism. Transfected cells that display a target phenotype identify the synthesized structures with desirable properties.

The present invention provides that a candidate genome is at least two strains of any sequenced viral genome, including, for example, pox virus. The present invention provides that a condition or disease in a target organism can be an inflammatory response, an autoimmune response, an organ-transplant rejection, a viral infection, a bacterial infection, a fungal infection, or some other condition. The present invention provides that the target organism to be treated can be mammalian or some other species of animal or even a plant.

The present invention provides a loop-end dynamic programming method comprising creating a two dimensional dynamic programming table to fit the window of the base sequence along a horizontal top of the two dimensional dynamic programming table and to fit the window of the base sequence along a vertical left side of the two dimensional dynamic programming table. Letters representing the base sequence of the window are placed, in order, starting at the 5 prime or 3 prime end, along the horizontal top of the two dimensional dynamic programming table, moving left to right, forming a horizontal base top. Letters representing the base sequence of the window are placed, in order, starting at an opposite end from the horizontal base top, along a vertical left side of the two dimensional dynamic programming table, moving top to bottom, forming a vertical base side. A table quality score is calculated for entry into each cell of a top-left half of the two dimensional dynamic programming table corresponding to each base-base interaction between the horizontal base top and the vertical base side base on the optimum pathway for matching. A score is calculated by adding a first number to an initial quality score for each A-U, U-A, C-G, or G-C base match, forming a cumulative score, adding a second number to the cumulative score for each G-U or U-G base match, adding a third negative number from the cumulative score for each 5 prime side bulge, adding a fourth negative number from the cumulative score for each 3 prime side bulge, and adding a fifth negative number from the cumulative score for a mismatch, wherein the mismatch is A-A, C-C, G-G, U-U, A-C, C-A, A-G, G-A, C-U, or U-C. The highest value in the table is located, and the corresponding stem-loop structure is stored. The present invention provides an alternative loop-end dynamic programming method wherein a score for entry into the table is calculated for the entire table rather than only in the top left diagonal or alternatively, in the lower right diagonal, which is mirror image of the top left diagonal. The present invention provides that for any stem-loop structure, the quality is calculated by adding a first number to an initial quality score for each A-U, U-A, C-G, or G-C base match, forming a cumulative score, adding a second number to the cumulative score for each G-U or U-G base match, adding a third negative number from the cumulative score for each 5 prime side bulge, adding a fourth negative number from the cumulative score for each 3 prime side bulge, and adding a fifth negative number from the cumulative score for a mismatch, wherein a mismatch is A-A, C-C, G-G, U-U, A-C, C-A, A-G, G-A, C-U, or U-C. The present invention provides that the initial quality score can be approximately zero, the first number can be approximately one, the second number can be approximately three-fourths, the third number can be approximately negative three, the fourth number can be approximately negative three, and the fifth number can be approximately negative three.

The present invention provides that the heterogeneity comprises measuring contiguous dinucleotide repeats and rejecting the stem-loop structures having approximately more than five contiguous dinucleotide repeats. The present invention provides that the measurement of the conservation comprises measuring repeats of stem-loop structures located in the candidate genome and rejecting the stem-loop structures having approximately zero repeats.

The present invention provides that the BLAST method comprises preparing a stem-loop structures data file for submission by formatting the stem-loop structures data file according to requirements of National Center for Biotechnology Information batch BLAST computer program, running the stem-loop structures data file on the batch BLAST computer program, and retrieving and storing an output data file from the batch BLAST computer program. The present invention provides that the parsing method comprises reading a NetBLAST output file from a computer readable medium, parsing the NetBLAST output file, and storing base sequence data when a base sequence of a candidate stem-loop structure has a base match of approximately 20 or more to a candidate genome.

The present invention provides that the phosphoramidite chemistry method comprises synthesizing a stem-loop structure using a Pol III RNA polymerase promoter on a chip array. The present invention provides that the assay method is a transcription factor reporter assay. The present invention provides that the target phenotype is cell survival after programmed cell death.

The present invention provides an RNAi composition, for treating a condition in a target organism comprising at least one type of stem-loop structure selected from the group consisting of SEQ ID NOs. 1-52 and combinations thereof. The present invention provides a pharmaceutical composition, for treating a condition in a target organism, comprising a composition composed of at least one type of stem-loop structure selected from the group consisting of SEQ ID NOs. 1-52 and combinations thereof and a pharmaceutically acceptable carrier.

The present invention provides a method for treatment of a condition in a target organism comprising administering an effective amount of a composition composed of at least one type of stem-loop structure selected from the group consisting of SEQ ID NOs. 1-52 and combinations thereof.

The present invention provides an RNAi composition for treating a condition in a target organism comprising a stem-loop structure or combinations thereof identified using any one of the identifying methods disclosed herein for identifying stem-loop structures. The present invention provides a pharmaceutical composition for treating a condition in a target organism comprising a pharmaceutically acceptable carrier and a stem-loop structure or combinations thereof identified using any one of the identifying methods disclosed herein for identifying stem-loop structures.

The present invention provides a method for treatment of a condition in a target organism comprising administering an effective amount of a stem-loop structure or combinations thereof identified using any one of the identifying methods disclosed herein for identifying stem-loop structures. The present invention provides a method for treatment of a condition in a target organism comprising administering an effective amount of a pharmaceutically acceptable carrier and a stem-loop structure or combinations thereof identified using any one of the identifying methods disclosed herein for identifying stem-loop structures.

One benefit of the present invention is that a means to quickly and efficiently screen any target genome for potential stem-loop structures without regard to thermodynamic or other limitations of art references is provided. Identified structures can be readily screened further to match a base sequence to a candidate genome. Further screening provides structures with demonstrated biological activity as displayed by phenotype.

Other embodiments of the present invention will be readily apparent to those skilled in the art based on the following detailed description, wherein embodiments of the present invention are described by way of illustrating the best mode for the invention. The invention is capable of other and different embodiments, and the details of the invention are capable of modifications by various means without departing from the spirit and scope of the present invention. In accordance, the drawings and the detailed description should be regarded as illustrative and not limiting.

### BRIEF DESCRIPTION OF THE DRAWINGS

FIG. 1 is a flow diagram of a method for identifying stem-loop sequences for use in treatment of a disease or condition by demonstration of in vitro phenotype attenuation after screening.

FIG. 2 is a flow diagram of a method in a data processing system for identifying stem-loop sequences from a candidate genome.

FIG. 3 is a flow diagram of a method for finding, storing, and calculating quality of a stem-loop sequence.

FIG. 4 is a flow diagram of a method for calculating quality of the method of FIG. 3.

FIG. 5 is a flow diagram of a method using an island and point and blunt folding for finding, storing, and calculating quality of a stem-loop sequence.

FIG. 6 is a flow diagram of a method for calculating quality of the method of FIG. 5.

FIG. 7 is a flow diagram of a method for parsing an output file from the matching program NETBLAST.

FIG. 8A-8H are a flow diagram providing an example of the method in FIG. 1.

FIG. 9 is a diagram of siRNA/viRNA synthesis.

FIG. 10 is a diagram of a loop-end dynamic programming table.

FIG. 11 is a diagram of an open-end dynamic programming table.

FIG. 12A is a table of pox virus putative viRNA's and shows the matching portions of the stem-loop sequences to the DNA sequences of homo sapiens.

FIG. 12B is a continuation of the table in FIG. 12A and shows the stem-loop sequences.

FIG. 13 is a chart of viRNA mediated survival based on acidity.

FIG. 14 is a table showing the forward and reverse oligos representing the stem-loop sequences of FIG. 12B and synthesized for transfection into cells.

### DETAILED DESCRIPTION OF THE INVENTION

Definitions

A data processing system is any desktop or other suitable computer system capable of running computer software in RAM or ROM.

An interfering stem-loop sequences or structures are RNA or DNA sequences that have significant complimentary base sequences such that such sequences have biological activity resulting from such complimentary structure.

A candidate genome is any genome from which interfering stem-loop sequences may be identified.

A condition or disease is any affliction.

A target organism is any organism of interest wherein a treatment of that organism is of interest.

A base sequence is any portion of a sequential RNA or DNA sequence of a candidate genome including the entire sequence.

Sequential overlapping windows contain the same number of consecutive bases of the candidate genome starting with a beginning point of the genome and incrementing by one base along the sequence to an end point of the genome. The beginning point and the ending point can include the entire genome sequence.

Consecutively bound base sequences are RNA or DNA bases sequences of the candidate genome from which interfering stem-loop structures may reside within a window.

An optimum base pairing is the pairing of bases within a particular window that provides the highest quality score.

A. Method Overview

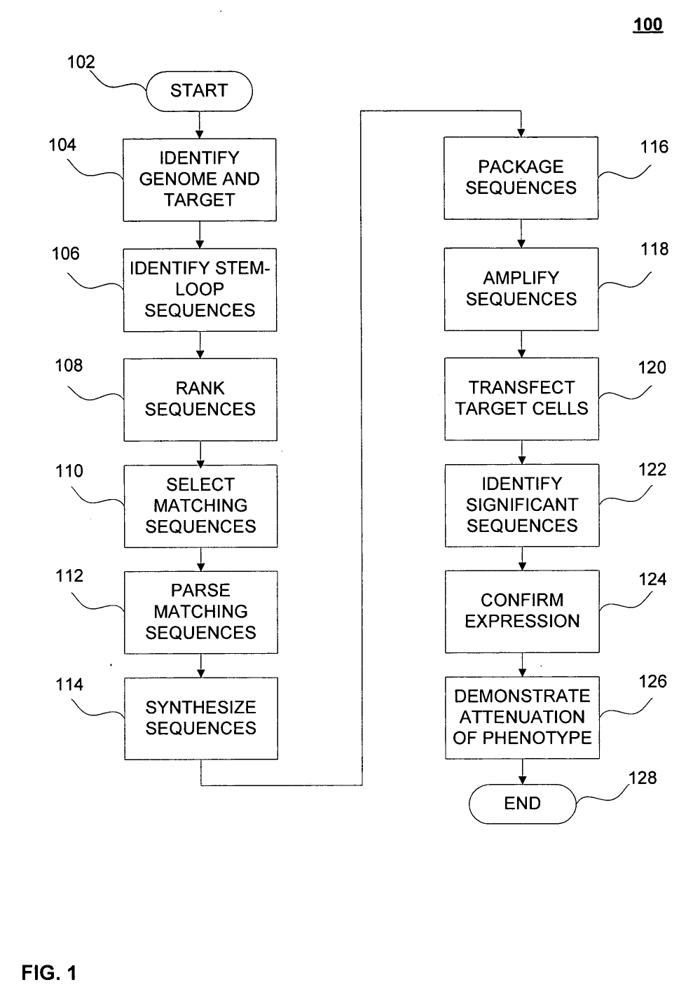

FIG. 1 is a flow diagram showing a method 100 for identifying interfering stem-loop sequences from a candidate genome for use in treatment of a condition or disease in a target organism in accordance with an embodiment of the present invention. First, the candidate genome and the target organism are selected 104. The interfering stem-loop sequences from the candidate genome are identified 106 using the method in a data processing system. Although the method could be performed manually, as a practical matter, a data processing system is best. The identified interfering stem-loop sequences are ranked 108 according to quality, heterogeneity, and conservation. After ranking, the interfering stem-loop sequences are matched 110 to the base sequence of the target organism in order to screen for those sequences with a higher likelihood of having biological activity in the target organism. In order to facilitate using the match sequences, such sequences are parsed 112 into a data file from which sorting for high scoring matching sequences can be performed. High scoring match sequences are synthesized 114 using phosphoramide chemistry. After synthesis, the sequences are packaged 116 in a retroviral library. PCR is used to amplify 118 the sequences, and the product is subcloned into a retroviral vector downstream of a Pol III promoter. The amplified sequences are transfected 120 into target cells, which are screened for function. Functionally significant sequences are identified 122 by PCR rescue. Northern blotting or DNA microaray is used to confirm expression 124 of sequences in transfected cells. A demonstration of attenuation of phenotype 126 in absence of sequences ends the method according to an embodiment of the present invention. One of skill in the art would readily understand that one can depart from the order of the method steps and the details of the methodology without departing from the spirit and scope of the above embodiment of the invention.

B. Identify Genome and Target

Referring to FIG. 1, the candidate genome is identified 104 to be at least two strains of a viral genome in one embodiment of the invention. Such a viral genome can be any sequenced viral genome and can include selection from any of the following viral families: “CrPV-like viruses”, “HEV-like viruses”, “SNDV-like viruses”, Adenoviridae, Allexivirus, Arenaviridae, Arteriviridae, Ascoviridae, Asfarviridae, Astroviridae, Baculoviridae, Barnaviridae, Benyvirus, Bimaviridae, Bomaviridae, Bromoviridae, Bunyaviridae, Caliciviridae, Capillovirus, Carlavirus, Caulimoviridae, Circoviridae, Closteroviridae, Comoviridae, Coronaviridae, Corticoviridae, Cystoviridae, Deltavirus, Filoviridae, Flaviviridae, Foveavirus, Furovirus, Fuselloviridae, Geminiviridae, Hepadnaviridae, Herpesviridae, Hordeivirus, Hypoviridae, Idaeovirus, Inoviridae, Iridoviridae, Leviviridae, Lipothrixviridae, Luteoviridae, Marafivirus, Metaviridae, Microviridae, Myoviridae, Nanovirus, Narnaviridae, Nodaviridae, Ophiovirus, Orthomyxoviridae, Ourmiavirus, Papillomaviridae, Paramyxoviridae, Partitiviridae, Parvoviridae, Pecluvirus, Phycodnaviridae, Picornaviridae, Plasmaviridae, Podoviridae, Polydnaviridae, Polyomaviridae, Pomovirus, Potexvirus, Potyviridae, Poxviridae, Pseudoviridae, Reoviridae, Retroviridae, Rhabdoviridae, Rhizidiovirus, Rudiviridae, Sequiviridae, Siphoviridae, Sobemovirus, Tectiviridae, Tenuivirus, Tetraviridae, Tobamovirus, Tobravirus, Togaviridae, Tombusviridae, Totiviridae, Trichovirus, Tymovirus, Umbravirus, Varicosavirus, and Vitivirus. The viral genome includes all members of the pox virus family. In another embodiment of the invention, the candidate genome can be any other sequenced genome.

The target organism can be identified 104 any animal or plant. The target organism can be any organism of interest for treatment using gene therapy based upon RNA or DNA type drugs. The condition for treatment in a target organism includes an inflammatory response, an autoimmune response, an organ-transplant rejection, a viral infection, a bacterial infection, and a fungal infection. Any condition that can be treated using gene therapy based upon RNA or DNA type drugs falls within the scope of the present invention.

C. Identifying Stem-Loop Sequences

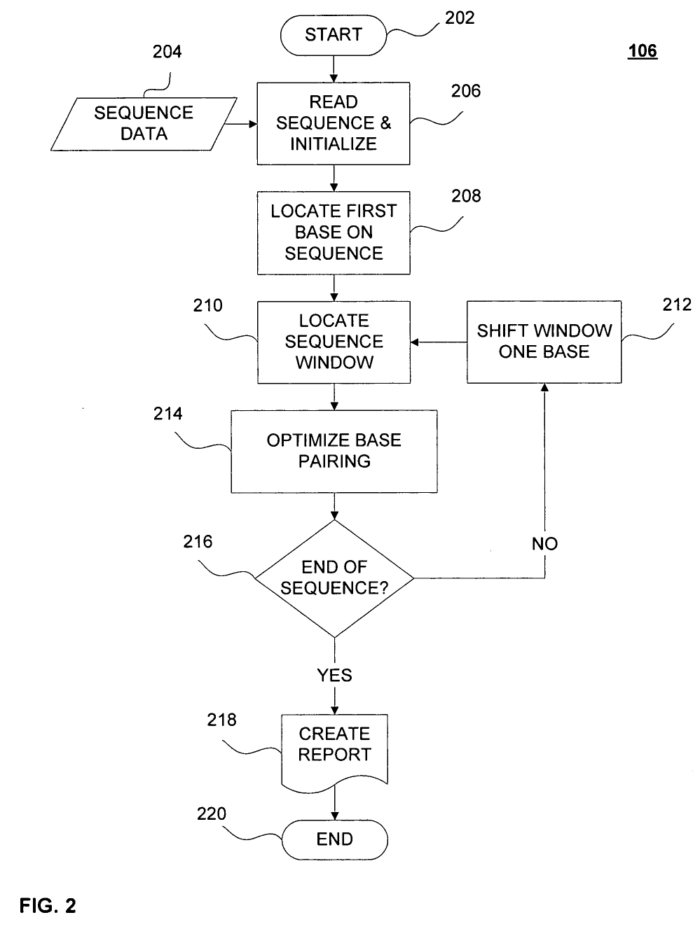

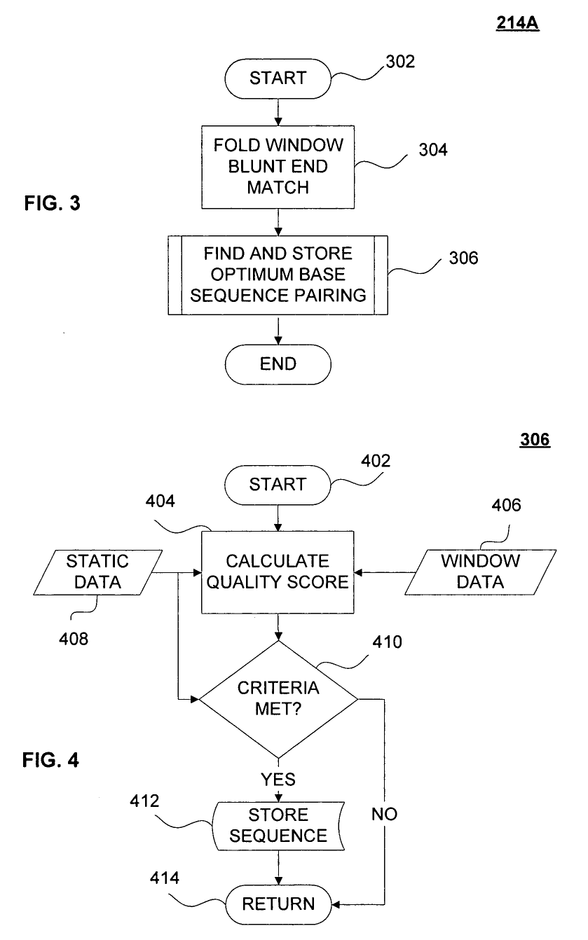

FIG. 2 is a flow diagram that shows the method of step 106 of method 100 in FIG. 1. Method 106 of FIG. 2 is performed in a data processing system and is for identifying interfering stem-loop sequences from a candidate genome for use in treatment of a condition or disease in a target organism. Referring to FIG. 2, the base sequence 204 of the candidate genome is read 206 from a computer readable medium into memory of the data processing system. Most commonly used desktop computer system can be used as the data processing system. Other computers systems may be suitable without departing from the scope of the invention. The computer readable medium may be a HD, CD, DVD, floppy disk, or other type of medium. The medium may reside on another computer system, which may be accessed through a network, including through a local Intranet or the Internet. A starting point along the base sequence is located to begin the search for interfering stem-loop sequences. Most preferably, the starting point is the first base 208 of the base sequence; however, another starting point could be chosen without departing from the scope of the invention. A loop begins 210 wherein a first window of sequential overlapping windows of base sequences is identified. The first window base pairings are then optimized 214. If the end of the base sequence is not reached yet 216, then the window is shifted by one base 212 to the next window. Each subsequent window undergoes optimum base pairing 214 until the end of the base sequence of the candidate genome is reached. An optimum base pairing for each sequential overlapping window is found by calculating a stem-loop quality using the dynamic programming method shown in FIG. 3 and FIG. 4. By way of illustration and without introducing limitations, if a base sequence was AGTTAAAATTTATAAAT GATTTACCAAAACTTGTCATCATATAAATTGATGGACCTAATGGAGTTATTATTGAGTTTATAAT T and if a window size was 20, the first window would be AGTTAAAATTTATAAATGAT, the second window would be GTTAAAATTTATAAATGATT, and so on. The last window would be TTATTATTGAGTTTATAATT. According to normal convention, A stands for adenine, C stands for cytosine, G stands for guanine, and T stands for thymine. Additionally, U stands for uracil and may be used interchangeable in sequence notation with the normal implication of representing a RNA sequence. The method ends after a report is created 218 from the optimized base pairings.

FIG. 3 is a flow diagram of the method of step 214 shown in FIG. 2. Method 214A of FIG. 3 is dynamic programming method. First, the window is folded 304 such that all base pairs are matched. Such folding is a conceptual device and is not necessary to practice the embodiment of the invention in FIG. 3. If the number of bases in the window is even, then all bases will be matched. Likewise, if the number of bases is odd, then there will be one unmatched base. An optimum base pairing is found and stored 306.

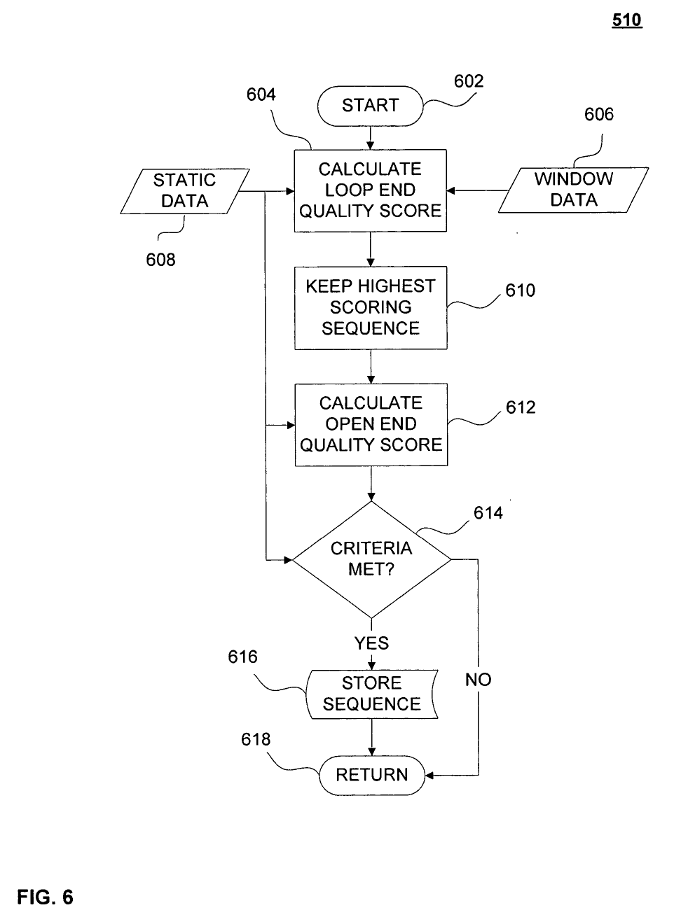

FIG. 4 is a flow diagram showing the method of step 306 in FIG. 3. FIG. 4 shows the method 306 for finding and storing the optimum base sequence pairing. Information about the window 406 and static program data 408 are provided to calculate a quality score 404 for each window. The static data 408 and window data 406 consist of the window sizes, the maximum loop size, the minimum loop size, the minimum stem length, the maximum stem length, the minimum seed island size, and numbers for base matches, partial-matches, mismatches, and stem bulges. The minimum seed island size represents consecutive bound base pairs in a folded window and is used in another embodiment of the invention shown in FIG. 6, step 608.

The maximum window size is preferably less than 200 bases, more preferably less than 160 bases, and most preferably 120 bases or less. The minimum loop size is preferably less than 10 bases, more preferably less than 6 bases, and most preferably 3 bases. The maximum loop size is preferably less than 70 bases, more preferably less than 55 bases, and most preferably 40 or less bases. The minimum stem length is preferably 10 or greater base pairs, more preferably 15 or greater base pairs, and most preferably 20 base pairs. The maximum stem length is preferably 35 or less base pairs, more preferably 30 or less base pairs, and most preferably 25 base pairs. The minimum seed island is preferably 8 or less base pairs, more preferably 5 or less base pairs, and most preferably 3 base pairs.

The match number for A-U, U-A, C-G, and G-C matches is preferably between 0.5 and 3.0, more preferably between 0.75 and 1.5, and most preferably 1.0. The partial-match number for G-U and U-G matches is preferably between 0.25 and 1.5, more preferably between 0.5 and 1.0, and most preferably 0.75. The five-prime bulge number for five prime side bulges is preferably −0.5 to −6.0, more preferably −1.5 to −4.5, and most preferably −3.0. The three-prime bulge number for three prime side bulges is preferably −0.5 to −6.0, more preferably −1.5 to −4.5, and most preferably −3.0. The mismatch number for base mismatches A-A, C-C, G-G, U-U, A-C, C-A, A-G, G-A, C-U, and U-C is preferably −0.5 to −6.0, more preferably −1.5 to −4.5, and most preferably −3.0.

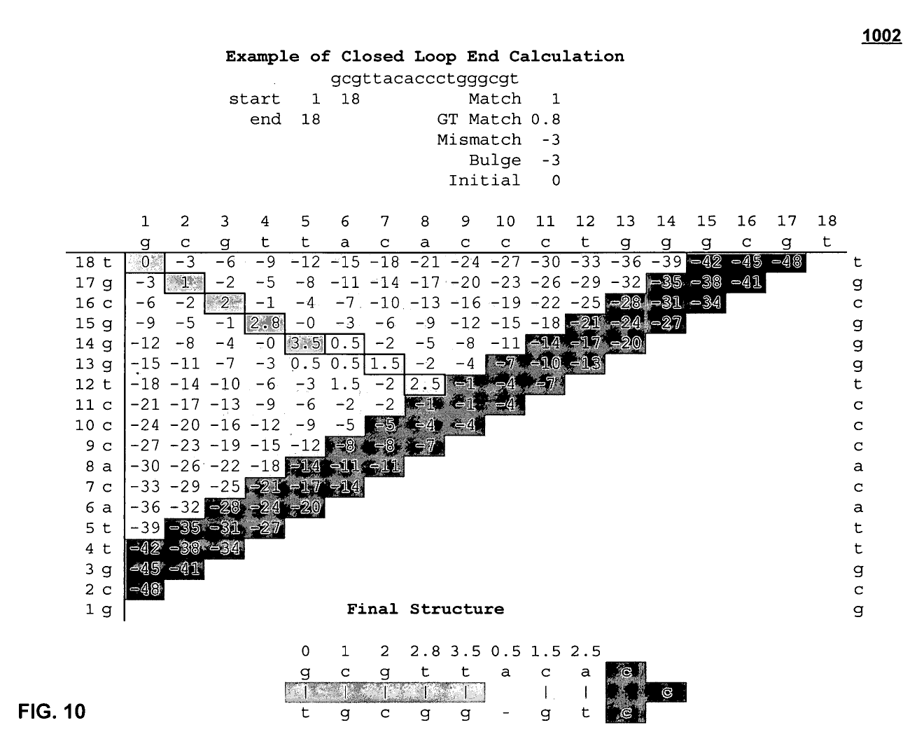

Method 306 of FIG. 4 further comprises a loop-end dynamic programming table method to calculate quality score 404. First, a two-dimensional dynamic programming table is created to fit the base sequence of a sequential overlapping window along a horizontal top of the two dimensional dynamic programming table and to fit the base sequence of the sequential overlapping window along a vertical left side of the two dimensional dynamic programming table. By way of illustration and without introducing limitations, if a base sequence were gcgttacaccctgggcgt, then the two-dimensional dynamic programming table would be as shown in FIG. 10. A table quality score is calculated for entry into each cell of the top-left half of the two dimensional dynamic programming table as shown in FIG. 10. The score corresponds to a cumulative base-base interaction between the horizontal base top and the vertical base side using a scoring method. The score calculation method is known in the computing art and is explained in Gusfield, D., Algorithms on Strings, Trees, and Sequences, Cambridge University Press, NY, 1997, the disclosure of which is incorporated by reference herein. The score is calculated by adding a match number to an initial quality score for each A-U, U-A, C-G, or G-C base match, forming a cumulative score, adding a partial-match number to the cumulative score for each G-U or U-G base match, adding a five-bulge number that is a negative number from the cumulative score for each 5 prime side bulge, adding a three-bulge number that is a negative number from the cumulative score for each 3 prime side bulge, and adding a mismatch number that is a negative number from the cumulative score for each A-A, C-C, G-G, U-U, A-C, C-A, A-G, G-A, C-U, or U-C mismatch. After completing the table, the highest value in the table is located. Such value corresponds to the optimum base pairing for the particular sequential overlapping window. Once obtained, the highest value and corresponding sequence of the sequential overlapping window are stored if the criteria are met. In one embodiment, suitable criteria are as follows: the highest value is approximately 10 or more, the maximum loop size is approximately 40 or fewer bases, the minimum stem length is approximately 20 or more base pairs, and the minimum loop size is approximately 3 or more bases when the window sizes is 120 bases. Depending on the window size, the cut-off point for the highest value in the table can range from approximately 5 to 15.

The scoring system gives positive value to beneficial structures like binding canonical base pairs or G-U mRNA binding, and negative value to disruptive structures like base mismatches or bulges. The score for a structure is the sum of all weights for the stem region. There is no weight based on loop size as long as it is less than the maximum allowed, and there is no provision for neighbor effects such as a different score for the case of two mismatches next to each other that is different from two-times the single mismatch weight. However, such provisions could be added and fall within the scope of the invention.

In an embodiment of the invention, the upper left corner of the table is set to the sequence one base loop side of the island, with the horizontal edge of the table corresponding to the 5prime portion of the entire loop sequence and the vertical edge of the table corresponding to the 3prime portion of the entire loop sequence. Because this problem is a folding and not just a matching problem, the table needs only to be filled to the northeast diagonal. The highest scoring structure is located, and the loop size counts for zero in the score. Therefore, once the DP table is filled-in using the structure scoring matrix weights, the highest scoring cell signals the best scoring structure. The maximum score path from the best cell to the upper left corner indicates what is the specific structure found. The algorithm keeps this structure by holding the new loop endpoints and a list of bulge base locations in a list. Two similar structures may be found that are spatially close on the target sequence. The algorithm will keep the highest scoring candidate when two candidates have similar loop center points or have similar stem start or end bases. Lower scoring candidates will be discarded. A similar structure is defined as structures having the same bases within approximately 6 bases, more preferably within 4 bases, and most preferably within 3 bases of each other.

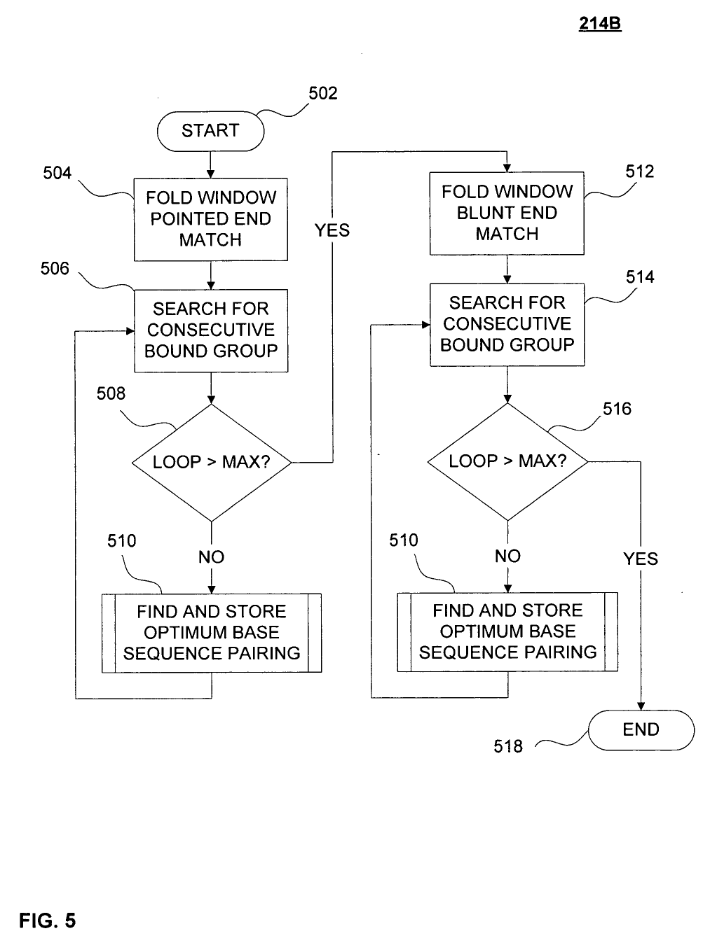

FIG. 5 shows method 214B that corresponds to step 214 of method 106 in FIG. 1. Method 214B is the preferred embodiment for base pair optimization. Method 214B is similar to method 214A but uses a base island anchor within the folded window in order to reduce calculation time. The basic parameters and corresponding ranges of method 214A apply to method 214B. To begin, a base sequence is folded 504 to form a pointed end match. As an example, if the window size were 119 bases, then folding would form 59 pairs of bases with one unpaired base at the fold. Such unpaired base is the pointed end of the folded window. Within the folded window, a search for a consecutive bound group 506 is performed starting from the loop-end. Such consecutive bound group is an island anchor of matching base pairs. As an example, an island for an RNA sequence could be comprised of -AUG- on one side of the fold and -UAC- on the other side of the fold, providing an A-U, U-A, and G-C paired matches within the island. Due to folding, the actual sequence of the window is -AUG- . . . -CAU- prior to folding. After an island is found, the method queries whether the loop size extend towards the loop end of the folded window exceeds the maximum loop size allowed. For example, if the window size were 119 with bases numbered consecutively, the maximum loop size were 40 base pairs, and the island were 3 base pairs, then an island matching bases 17, 18, and 19 to 103, 102, and 101 respectively would fall outside the 40 base pair limit by one base pair grouping. If the loop size maximum is not exceeded, then the method finds and stores the optimum base sequence pairing 510 incorporating the island.

FIG. 6 shows method 510 of FIG. 5. Similar to method 306, static data 608 and window data 606 are input to calculate quality score 604, 612. The folded window with the island will have an open-end and a loop-end. An optimum base sequence pairing for each island window is calculate by calculating a loop-end quality 604 in a loop-end region of the consecutively bound base pair grouping using a loop-end dynamic programming table method and calculating an open-end quality 612 in an open-end region of the consecutively bound base pair grouping using an open-end dynamic programming table method. The loop-end quality is calculated using the loop-end dynamic programming table method of method 306, step 404 of FIG. 4. The open-end dynamic programming table method is the same as method 306, step 404, except that the entire table is completed. Once a loop-end score and open-end score are calculated, the found structure is compared to criteria and stored if the criteria are met. In one embodiment, suitable criteria are as follows: the highest value is approximately 10 or more, the maximum loop size is approximately 40 or fewer bases, the minimum stem length is approximately 20 or more base pairs, and the minimum loop size is approximately 3 or more bases when the window sizes is 120 bases. Depending on the window size, the cut-off point for the highest value in the table can range from approximately 5 to 15. The score for a structure is the sum of all weights for the stem region. There is no weight based on loop size as long as it is less than the maximum allowed. There is no provision for neighbor effects, wherein a different score for the case of two mismatches next to each other that is different from two-times the single mismatch weight. However, such provisions could be added and fall within the scope of the invention.

The search for highest score on the loop side of the seed island uses a modified dynamic programming (DP) technique. The upper left corner of the DP table is set to the sequence one base loop side of the island, with the horizontal edge of the table corresponding to the 5prime portion of the entire loop sequence and the vertical edge of the table corresponding to the 3prime portion of the entire loop sequence. Because this problem is a folding and not just a matching problem, the DP table needs only to be filled to the northeast diagonal.

The highest scoring structure is located, and the loop size counts for zero in the score. Therefore, once the DP table is filled in using the structure scoring matrix weights, the highest scoring cell signals the best scoring structure. For tied scores, the first score is kept. The maximum score path from the best cell to the upper left corner indicates what is the specific structure found. The algorithm keeps this structure by holding the new loop endpoints and a list of bulge base locations in a list.

The highest scoring loop side structure found above is now extended toward the open end using a very similar algorithm as above. The upper left cell represents one base beyond the open side of the seed island, with the horizontal edge containing a reverse sequence of the 5prime strand and the vertical edge containing a reverse sequence of the 3prime strand. The same scoring matrix is used, but because of the open end, the entire rectangular table must be filled.

Similarly to the loop side search, the highest scoring structure is located, so the maximum cell again provides the starting point for the maximum score path back to the upper left corner. When finding the path back to the upper left corner, equal scores are defaulted in the order mismatch, 3prime bulge, and lastly 5prime bulge. This algorithm keeps this found structure by holding the new stem endpoints and adding any bulge base locations to the list produced by the loop side search algorithm.

Two similar structures may be found from islands, which are spatially close on the target sequence. The algorithm will keep the highest scoring candidate when two candidates have similar loop center points or have similar stem start or end bases. Lower scoring candidates will be discarded. Similar loop center points is currently defined as being within 3 bases of each other for any of the above three structure characteristics.

After the loop size exceeds the maximum 508, the window is refolded 512 to match all base pairs with a blunt end at the loop-end. A blunt end means that all bases are paired right up to the end of the loop. The search for an island group is repeated 514. Once an island is found, the loop size is check against the maximum 516 as before. If the loop size is less than the maximum, then the optimum base sequence and quality score are found and stored 510 as before with the pointed end match folding of the window.

The folding of a window in the aforementioned dynamic programming methods is conceptual and is not limiting. The dynamic programming method performs the matching optimization according to the matching criteria regardless of conceptual folding.

D. Rank Sequences

Sequences from method 106 are sorted and ranked according to the highest scoring sequences. The preferred embodiment is to the sorting feature of any commercially available spreadsheet type computer program such as Microsoft Excel®.

E. Select Matching Sequences

Step 110 of method 100 involves a simple way to identify single or multiple candidate target genes by screening output of step 108 (i.e. the putative viRNA's/stem-loop structures) against an expressed sequenced tags (EST) database, corresponding to the host organism, to determine whether there are significant matches between the viRNA's and the host genes. Step 106 produces an output that represents only the stem-loop structure, with no flanking sequences, which means submission to the EST database screening algorithm is straightforward. To facilitate this screening, these viRNA sequences are screened in batch mode using the pre-existing BLASTc13 tool from the National Center for Biotechnology Information (NCBI) that provides an automated method for homology searching for a large number of sequence queries Altschul, S. F., Gish, W., Miller, W., Myers, E. W. & Lipman, D. J. (1990) “Basic local alignment search tool.” J. Mol. Biol. 215:403-410. To further understand the quality of this data, the stochastic “noise” of identifying false viRNA's from steps 106, 108, and 110 can also be measured by comparing the output with the random sequences used to validate the performance of step 106.

F. Parse Matching Sequences

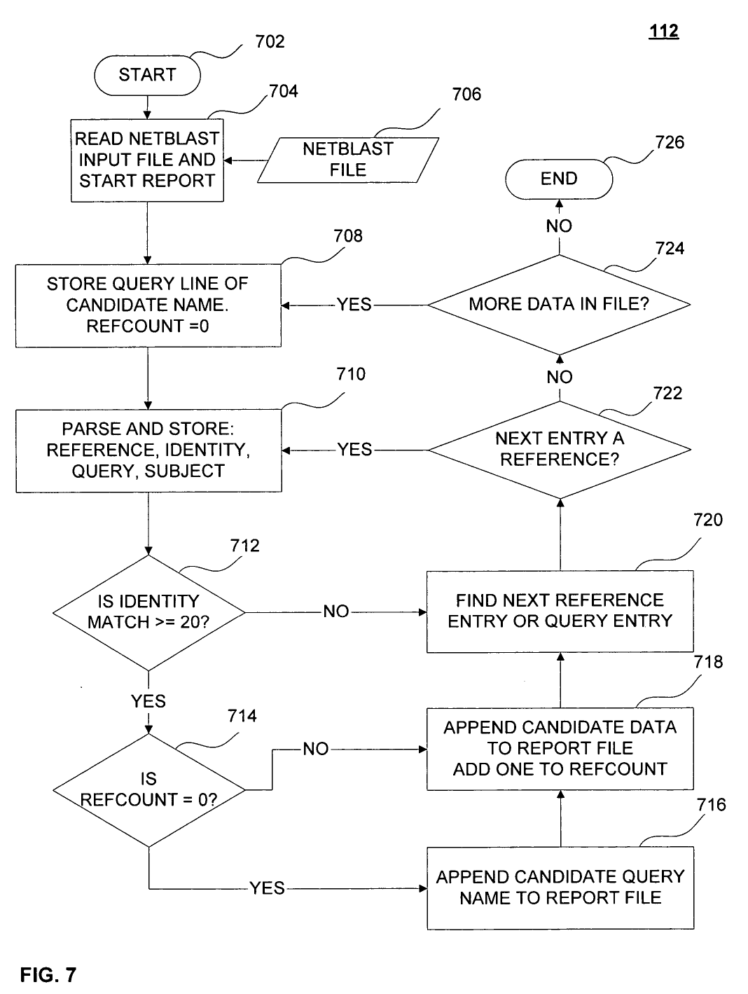

Step 112 of method 100 involves a “BLAST” output that can be large and difficult to understand, depending on the number of stem-loops screened and the number of “hits” identified in a blast search. As an example with poxvirus genomes, the text file output exceeded 45 Mb due to the presence of common repeat sequences. Therefore, a text parser was designed that can filter the output to identify the high quality matches (with a low BLAST “E” value and high identity) and matches that correspond to the stem region, rather than the loop region. FIG. 7 shows method 112 for parsing a BLAST output file. A NetBlast file 706 is read and a report started 704. The query line of the candidate name is stored 708. Reference, identity, query, and subject are parsed and stored 710. The identity match is checked against the stem-length size 712. As an example, step 712 shows a stem size of 20 base pairs. If the stem size is 20 or greater, then step 714 checks to see if the candidate has been located previously or not. Step 716 appends the candidate name to the report if new. Step 718 appends the candidate data to the report file. Step 720 locates the next entry. Step 722 determines whether the next entry is a reference. Step 724 looks for more data in the file if the next entry was not a reference.

G. Synthesize Sequences

Step 114 of method 100 involves synthesis of viRNA. Representative United States patents that teach synthesis of polymers and nucleic acids include the following: U.S. Pat. Nos. 6,456,942; 6,444,111; 6,280,595; and 6,093,302. The disclosure of each patent is incorporated by reference herein. De novo RNA synthesis is considerably more expensive than DNA oligonucleotide synthesis. In order to economically screen a larger number of viRNA molecules, a multi-step process is used to synthesize the RNA candidates, which allows easy quality control and permits us to have a virtually inexhaustible supply of material. This approach has similarities to other approaches used for high throughput screening of siRNA molecules and is well established. Example approaches are taught by Gou D., Jin N., Liu L., Gene silencing in mammalian cells by PCR-based short hairpin RNA, FEBS Lett. 2003 Jul. 31; 548(1-3):113-8 and Sohail M, Doran G, Riedemann J., Macaulay V., Southern E. M., A simple and cost-effective method for producing small interfering RNAs with high efficacy, Nucleic Acids Res. 2003 Apr. 1; 31(7), which are incorporated by reference herein. Using this method provides cloning the viRNA templates after the PCR reaction to use as an expression vector-based system, as an alternative to the direct transfection of transcribed viRNA's. Example approaches are taught by Arts G J, Langemeijer E, Tissingh R, Ma L, Pavliska H, Dokic K, Dooijes R, Mesic E, Clasen R, Michiels F, van der Schueren J, Lambrecht M, Herman S, Brys R, Thys K, Hoffmann M, Tomme P, van Es H. Adenoviral vectors expressing siRNAs for discovery and validation of gene function, Genome Res. 2003 October; 13(10):2325-32, which is incorporated by reference herein.

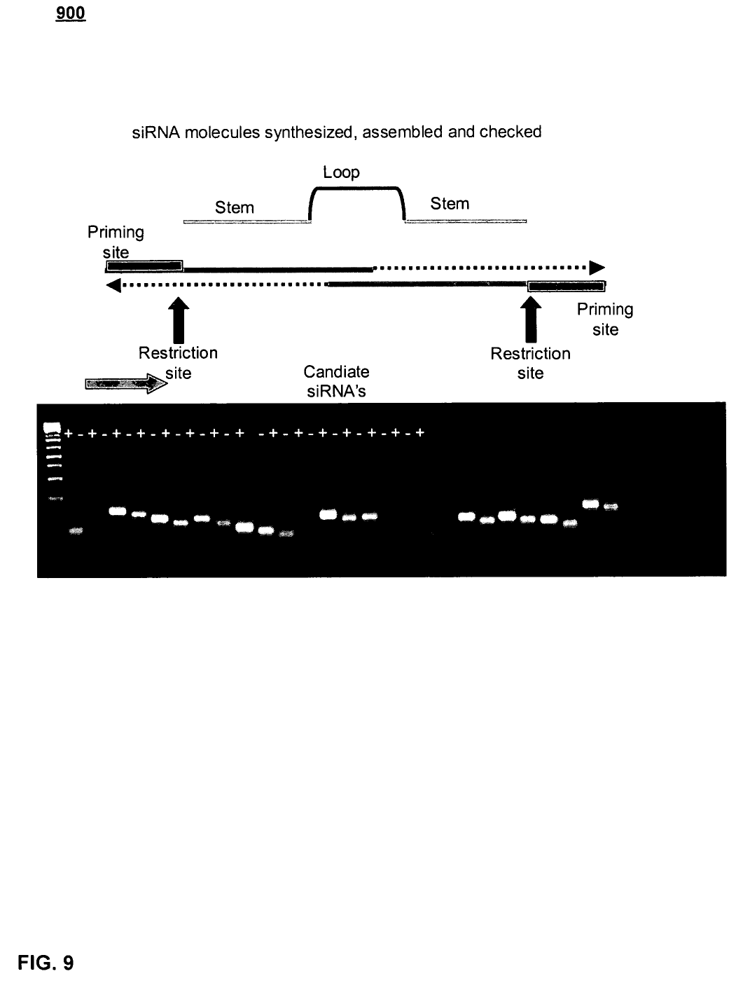

An expression vector-based system may be used. Generating viRNA's from DNA oligonucleotides is established and has been used routinely for the generation of validated reagents for post-transcriptional gene silencing. A two-oligo approach on opposing stands is used as a method to lower the risk of N-1 deletions during phosphoramidite synthesis producing mutations in the viRNA molecule. In addition, this method prevents problems during phosphoramidite synthesis due to hairpin formation within a single oligonucleotide. An RNA polymerase (T7) promotor site is incorporated into the left hand primer (below). The oligos are annealed in the region corresponding to the mismatched loop and then extended to generate a double-stranded DNA template that encodes the viRNA. The product is then amplified by PCR, and one end truncated by restriction digestion with Mly-1. The double-stranded PCR product is transcribed in vitro to produce the corresponding RNA sequence. After transcription, the DNA template is removed by DNase I digestion and purification. The RNA is checked and quantified, then self-annealed just prior to transfection. FIG. 9 shows a method for viRNA or siRNA synthesis.

H. Package Sequences

Step 116 of method 100 involves packaging of the sequences. A commercially suitable packaging system is provided by BD Biosciences, entitled Retro-X System or RetroXpress System. Representative articles that teach packaging a cloned retroviral library in a suitable packaging line include the following: Coffin, J. M., et al. (1996) Retroviruses (CSHL Press, NY); Ausubel, F. M. et al. (1996) Current Protocols in Molecular Biology (John Wiley & Sons, NY), Supplement 36, Section III; Mann, R., et al. (1983) Cell 33:153-159; Miller, A. D. & Buttimore, C. (1986) Mol. Cell. Biol. 6: 2895-2902; Morgenstern, J. P. & Land, H. (1990) Nucleic Acids Res. 18:3587-3596; Miller, A. D. & Chen, F. (1996) J. Virol. 70:5564-5571; Miller, D. G. & Miller, A. D. (1994) J. Virol. 68: 8270-8276; Miller, A. D. (1996) Proc. Natl. Acad. Sci. USA 93: 11407-11413; Miller, A. D. & Rosman, G. J. (1989) BioTechniques 7:980-990; and Miller, D. G., et al. (1994) Proc. Natl. Acad. Sci. USA 91:78-82. The disclosure of each article is incorporated by reference herein.

I. Amplify Sequences

Step 118 of method 100 involves amplifying the sequences. The viRNA's can either be screened individually or screened in batch mode. viRNA's are cloned into retroviruses, or other viral vectors that integrate into the host genome. Batch mode means that multiple viRNA encoding retroviruses would be combined and screened simultaneously for phenotype. Cells are infected with the viRNA encoding retrovirus then those cells that exhibit the desired phenotype are segregated. Genomic DNA from the cell is isolated, and then retrovirus specific primers are used to amplify the viRNA sequence from the cells with the desired phenotype using standard recovery methods. A representative article that teaches screening includes the following: Wong B Y, Chen H, Chung S W, Wong P M. High-efficiency identification of genes by functional analysis from a retroviral cDNA expression library. J. Virol. 1994 September; 68(9):5523-31, the disclosure of which is incorporated by reference herein.

J. Transfect Target Cells

Step 120 of method 100 involves transfecting target cells of the host. As an example, results using a screen of the poxvirus genome identified a number of putative viRNA motifs, of which 13 were screened in the apoptosis assay and compared to a panel of caspase siRNA controls. Biological models for determining viRNA function are straightforward cell-based assays, with simple endpoints, to allow screening large numbers of viRNA candidates by transfection. Two primary phenotypes were investigated: Apoptosis inhibition and Activation of an inflammatory transcription factor pathway.

For apoptosis inhibition, across the viral phylum there are numerous virally encoded proteins that have been shown to inhibit the apoptotic process of the host cell. Presumably these functions have been selected for since they prevent destruction of the virally infected host cell by the host immune system or by an autonomous mechanism and allow the virus to continue to replicate in the host. A hypothesis is that it may be possible to identify multiple anti-apoptotic viRNA candidates. Data using a pox virus viRNA is provided. These anti-apoptotic viRNA's are interesting because cell death is thought to be a significant part of patho-physiological processes in certain autoimmune disorders and additional insight into novel mechanisms for inhibiting this pathway would be valuable. The cell death/apoptosis assay was carried out in the human E1A transformed embryonic kidney cell line, 293, which can be efficiently transfected. Established treatments of 20 ng/ml Tumor Necrosis Factor alpha and 10 micrograms/ml cycloheximide or FAS activation through antibody cross-linking to induce apoptosis was used. A representative article that teaches treatments is White, E., P. Sabbatini, M. Debbas, W. S. M. Wold, D. I. Kusher, and L. R. Gooding, 1992, The 19-kilodalton adenovirus E1B transforming protein inhibits programmed cell death and prevents cytolysis by tumor necrosis factor, Mol. Cell. Biol. 12:2570-2580, the disclosure of which is incorporated by reference herein. These assays were controlled to determine whether the viRNA's are toxic without additional stimuli, as seen for a subset of viRNA's in other experiments.

Some viRNA's may have anti-inflammatory potential. Since different viRNA molecules may act in distinct areas of the inflammatory signaling cascade, a universal endpoint or “reporter” of inflammatory activation is required. Since transcription factor activation is often a sequelae of inflammatory pathway signaling, a transcription factor reporter assay is used to measure transcriptional activation or repression in response to an inflammatory stimuli, such as tumor necrosis factor alpha. Candidate viRNA's were tested for their ability to represses or activates the transcription factor NF-Kappa B in a reporter assay. NF kappa B was chosen as a reporter since it is at the heart of most inflammatory responses and also plays a role in the apoptotic machinery of the cell. Established reporter essay approach was used to determine the state of NF-kappa B mediated transcription in the presence of candidate viRNA's or controls. A reference that teaches established reporter assay approach is Mitchell T, Sugden B. J Stimulation of NF-kappa B-mediated transcription by mutant derivatives of the latent membrane protein of Epstein-Barr virus. Virol. 1995 May; 69(5): 2968-2976, the disclosure of which is incorporated by reference herein.

K. Identify Significant Sequences

Step 124 of method 100 involves confirming the expression. This involves confirming viRNA's with an anti-apoptotic or NF-kappa B repressing phenotype activity. A microarray-based approach was used to assay the extent and specificity of post-transcriptional gene silencing. A representative article that teaches this approach is Williams N S, Gaynor R B, Scoggin S, Verma U, Gokaslan T, Simmang C, Fleming J, Tavana D, Frenkel E, Becerra C., Identification and validation of genes involved in the pathogenesis of colorectal cancer using cDNA microarrays and RNA interference, Clin Cancer Res. 2003 March; 9(3):931-46, the disclosure of which is incorporated by reference herein. Another approach is to use the Combimatrix “CustomArray902” product. This system allows rapid design of a series of microarrays directed against any genes of interest in any system. Using the ability to identify possible host targets of the viRNA molecules based on sequence homology, such information can be used to develop a custom microarray most specific to those genes that are most likely to be the targets of viRNA inhibition. The viRNA's with the most significant activity in the apoptosis and NF-kappa B assays were selected. These viRNAs were transfected and after 72 hours the mRNA of viRNA treated cells (versus cells with a control transfection) were purified, fluorescently labeled and hybridized to a microarray to determine the extent of post-transcriptional gene silencing. Using this approach, identification of the transcripts that are being targeted by viRNA molecules for PTGS was done. The PTGS mediated by the viRNA may be entirely responsible for the phenotype. Classical siRNA approach may be used to inhibit the same target(s). Using a panel of siRNA directed to the same host gene mRNA's as the viRNA, provides significant credence to show that the viRNA mediates its biological effect purely through an inhibitory RNA mechanism.

L. Demonstration of Attenuation of Phenotype

Step 126 of method 100 involves demonstration of phenotype. The effects of viRNA's can be characterized using standard assays for biological function comparing an unknown viRNA with a negative control (a stem-loop RNA with no homology to human transcribed RNA sequences) and a positive control (a stem-loop RNA that has a high level of stem homology with a gene known to be directly related to the phenotype being studied) in an in vitro or in vivo assay. Assays for determining the effects of a perturbing agent are widely described since this is the same sort of assay that would be used to assess the efficacy of a small molecule drug, antisense compound or gene knockout phenotype. Specific examples of the assays employed to asses the effect of a viRNA include:
cell-death/apoptosis assays, where cellular viability is assessed under a variety of lethal stimuli (see White E, Sabbatini P, Debbas, M, Wold W S M, Kusher D I, Goodling L R (1992) The 19 kilodalton adenovirus E1B transforming protein inhibits programmed cell death Mol Cell Biol 12:2570-2580);
transcription factor reporter assays (tools and methods for these techniques are described in de Wet, J. R., K. V. Wood, M. DeLuca, D. R. Helinski, and S. Subramani. 1987. Firefly luciferase gene: structure and expression in mammalian cells. Mol. Cell. Biol. 7:725-737. King, P., and S. Goodbourn. 1994. The -interferon promoter responds to priming through multiple independent regulatory elements. J. Biol. Chem. 269:30609-30615. King, P., and S. Goodboum. 1998. STAT1 is inactivated by a caspase. J. Biol. Chem. 273:8699-8704 Masson, N., M. Ellis, S. Goodbourn, and K. A. W. Lee. 1992. Cyclic-AMP response element-binding protein and the catalytic subunit of protein kinase A are present in F9 embryonal carcinoma cells but are unable to activate the somatostatin promoter. Mol. Cell. Biol. 12:1096-1102 Muzio M, Saccani S. TNF signaling: key protocols. Methods Mol Med. 2004; 98:81-100;
(1) microarray, SAGE or bead based methods for assessing changes at the RNA level;
ELISA, western blot, mass spectrometric methods for assessing changes in protein expression level or characteristics of the protein such as alterations in post transcriptional modification; and
changes in metabolite concentration measure by mass spectrometric methods.

M. Example

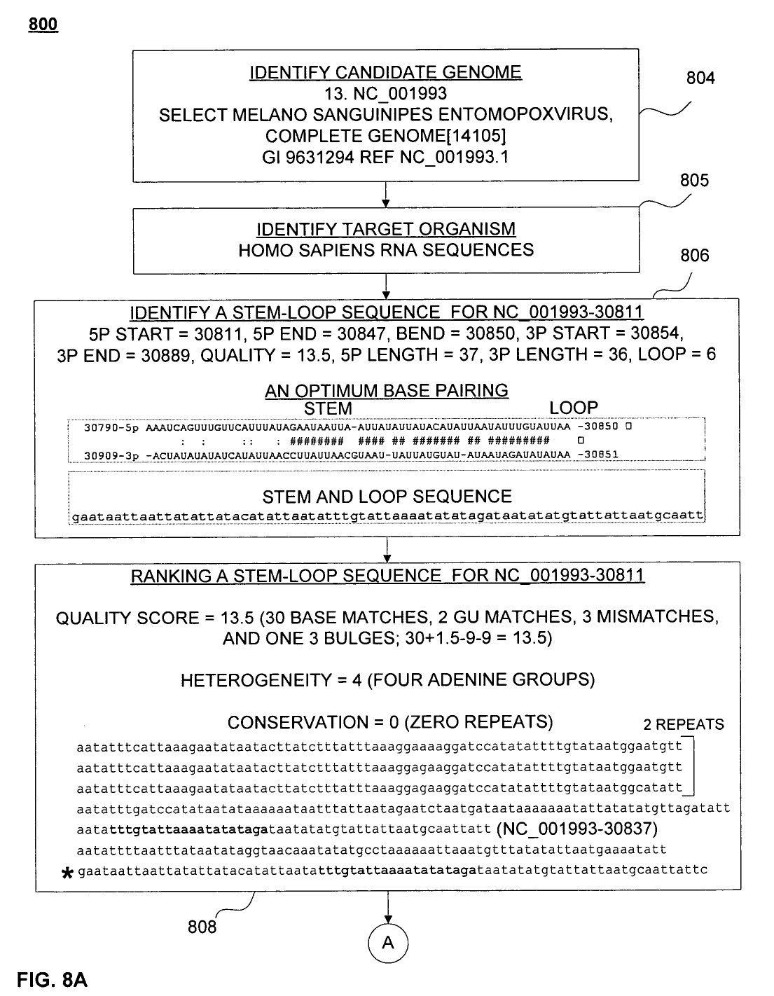

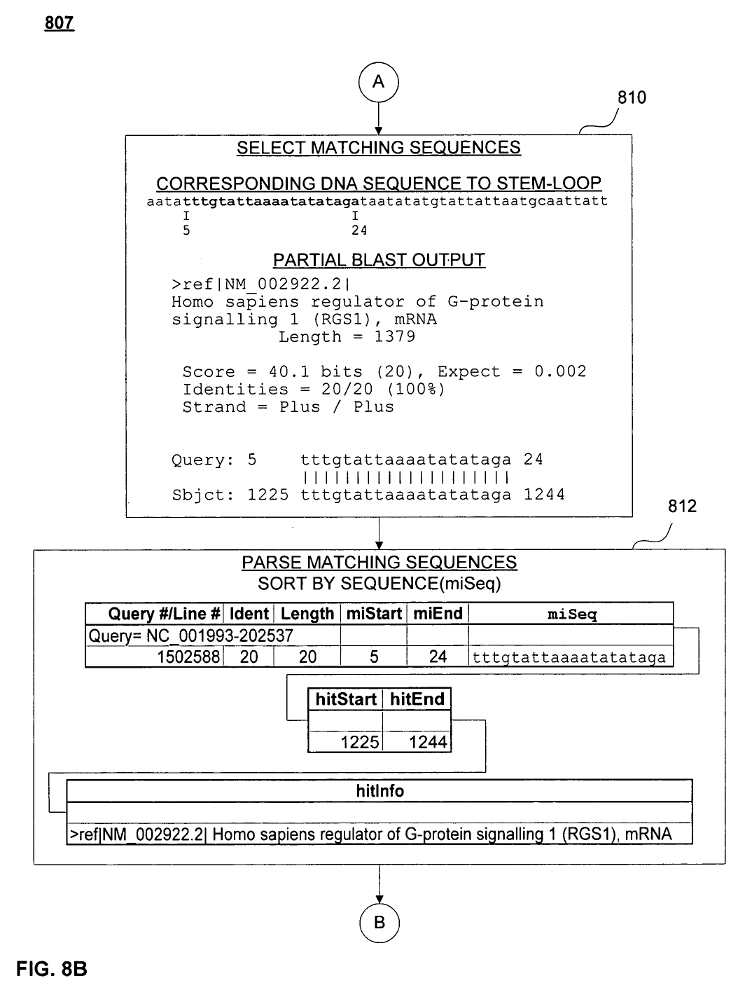

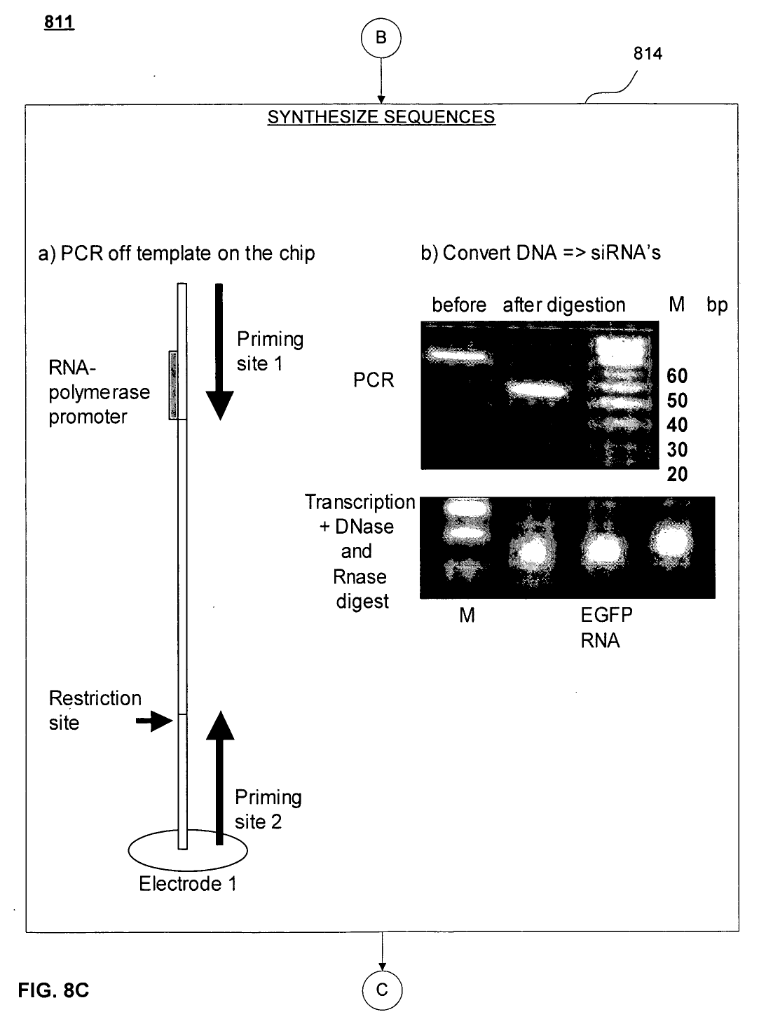

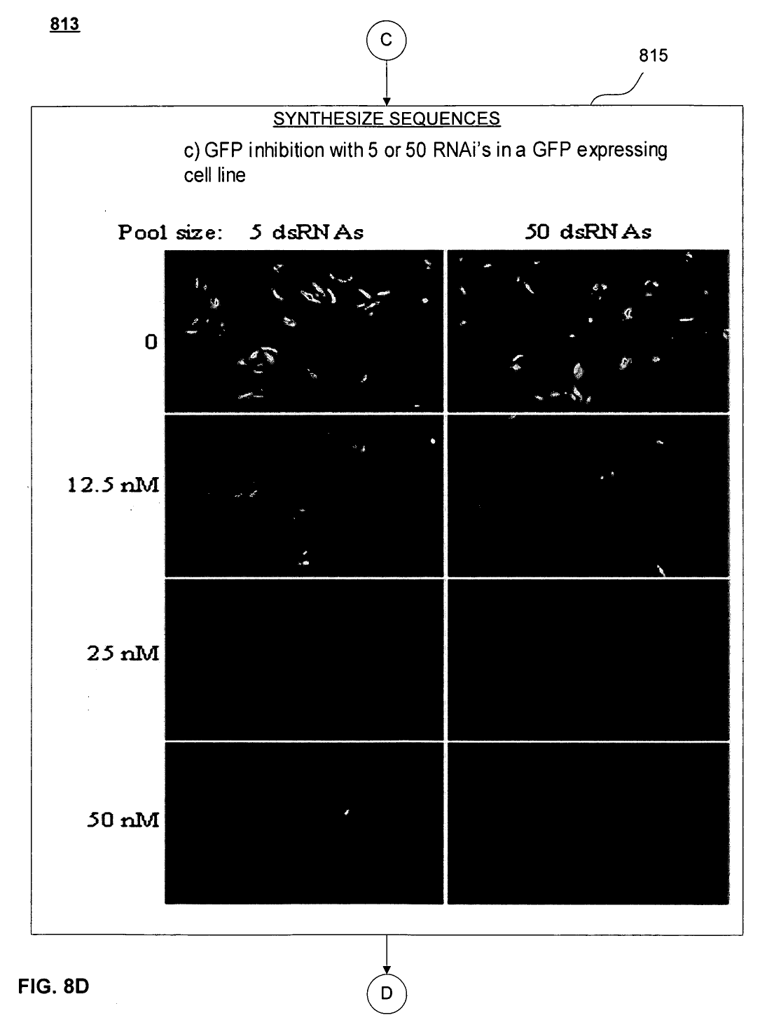

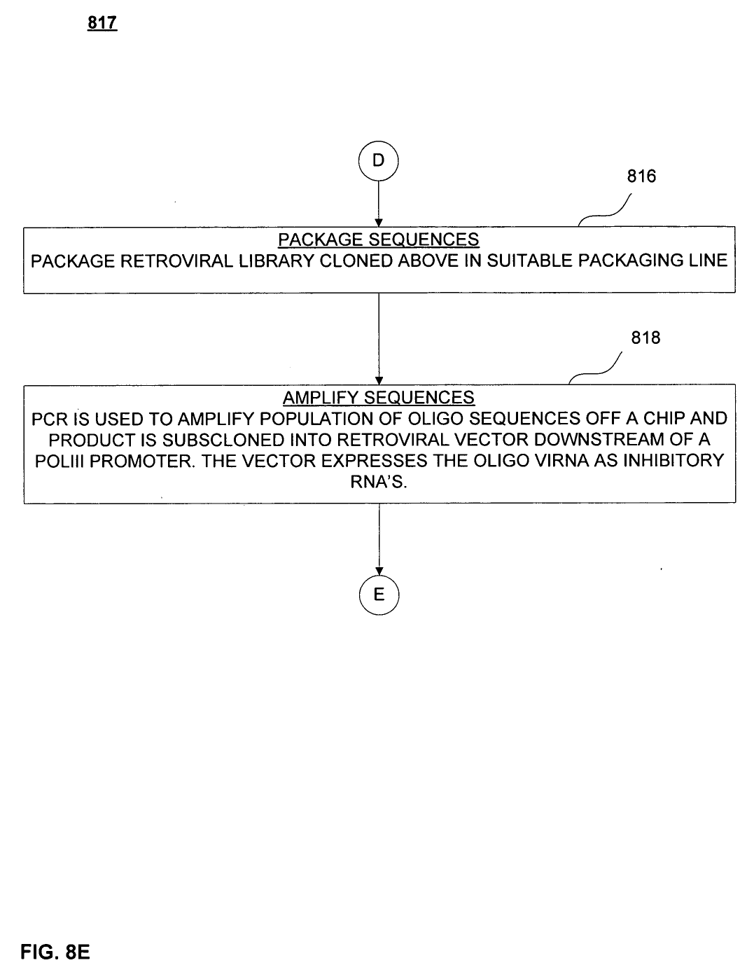

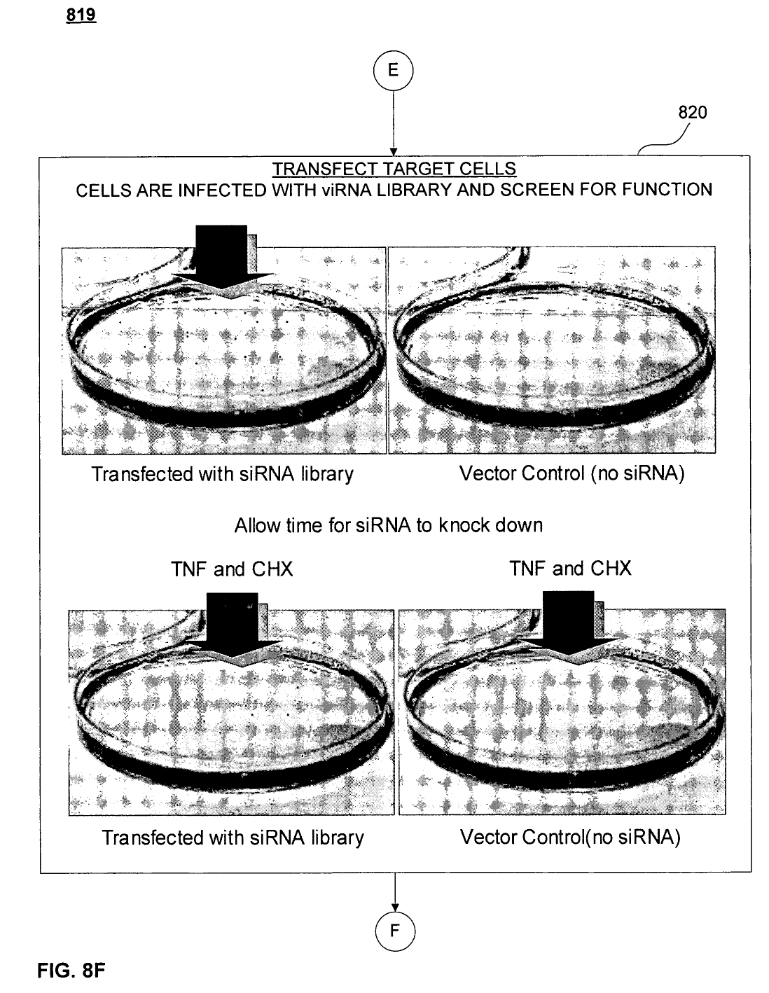

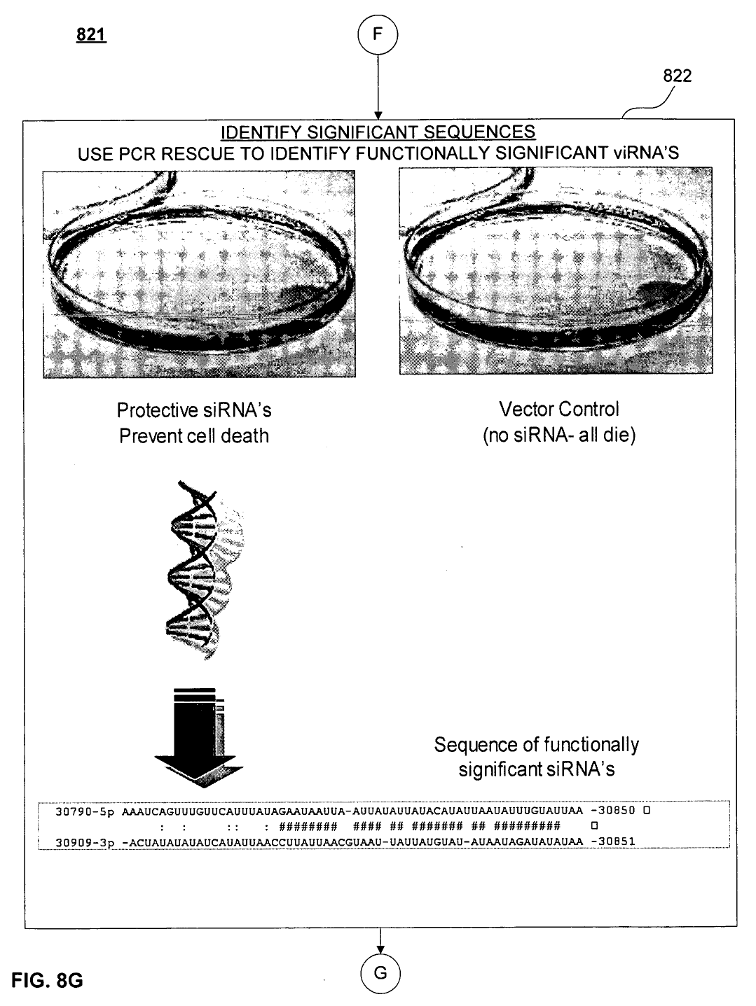

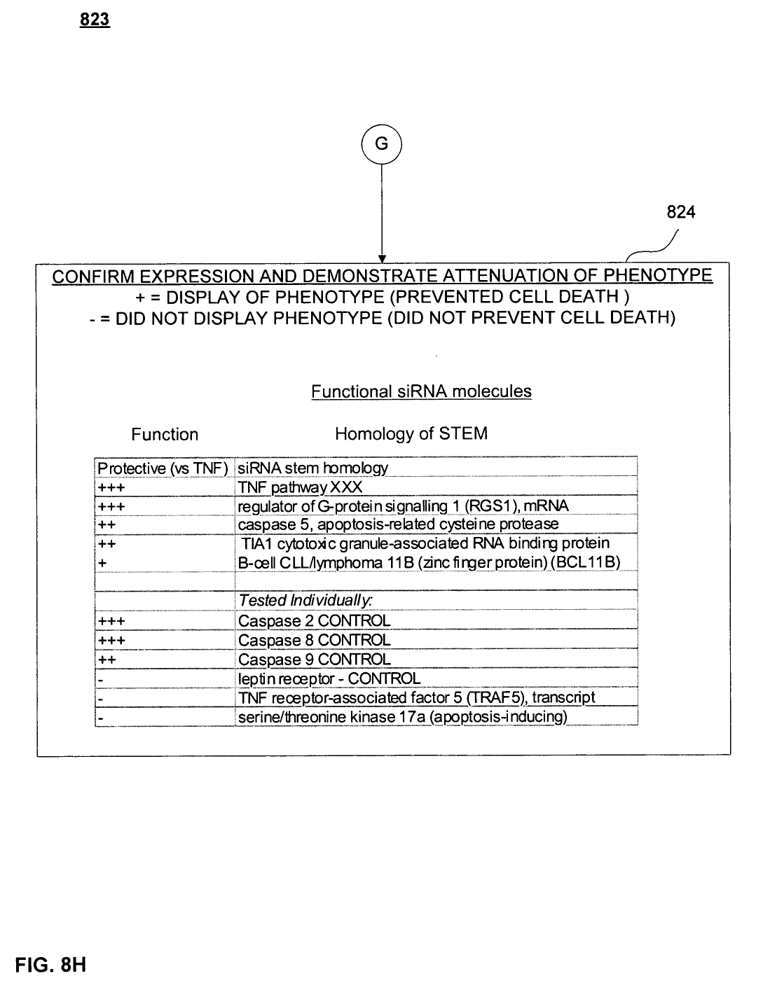

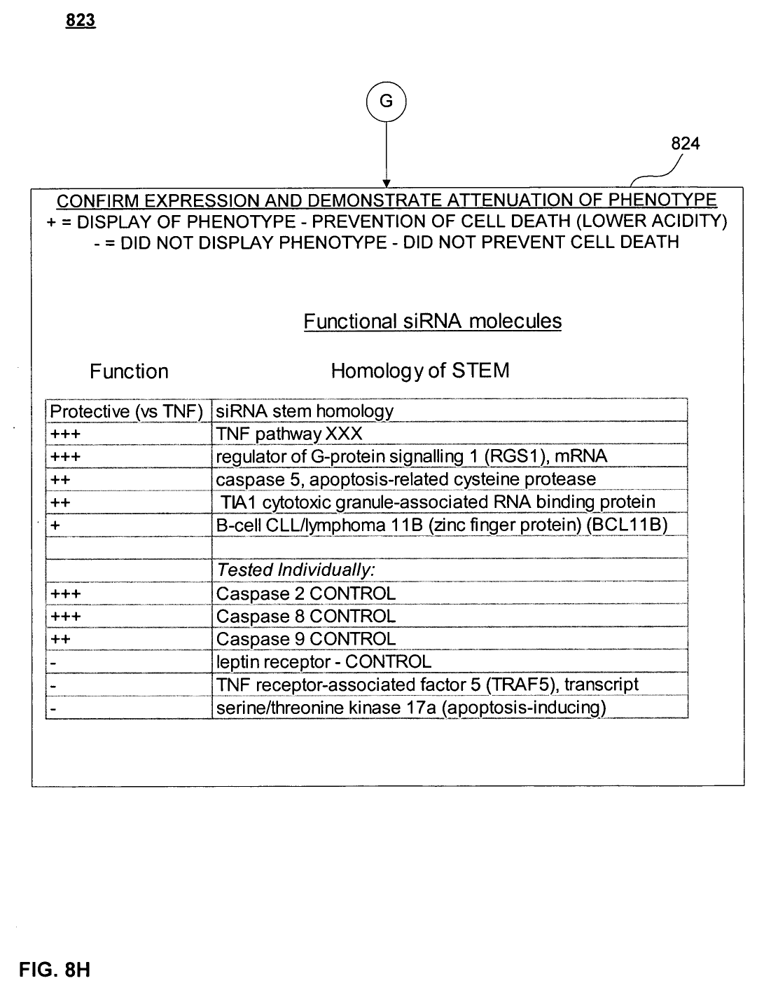

FIGS. 8A through 8H show an example of an embodiment of the present invention for identifying an interfering stem-loop structure within a candidate genome for treatment of a disease or condition in a target organism. Step 804 of method 800 shows selection of meloan sanguinipes entomopoxvirus as the candidate genome. Step 805 shows selecting homo sapiens as the target organism. Step 806 shows an optimum base pairing for a sequence starting at base 30,790 and ending at base 30,909. Thus, the window size is 120 bases. The five prime starting base for the stem is 30811. The five prime ending base for the stem is 30847. The loop or bend is at base 30850. The three prime starting point for the base from the loop side is 30854. The three prime ending base is 30889. The number of bases on the five prime side of the stem is 37. The number of bases on the three prime side of the stem is 36. There are three mismatches, three bulges, 30 matches, and two partial matches. If the scoring matrix is −3 for mismatches and bulges, 0.75 for partial matches, and 1 for matches, then the score is 13.5. The resulting stem-loop sequence is shown written in DNA format.

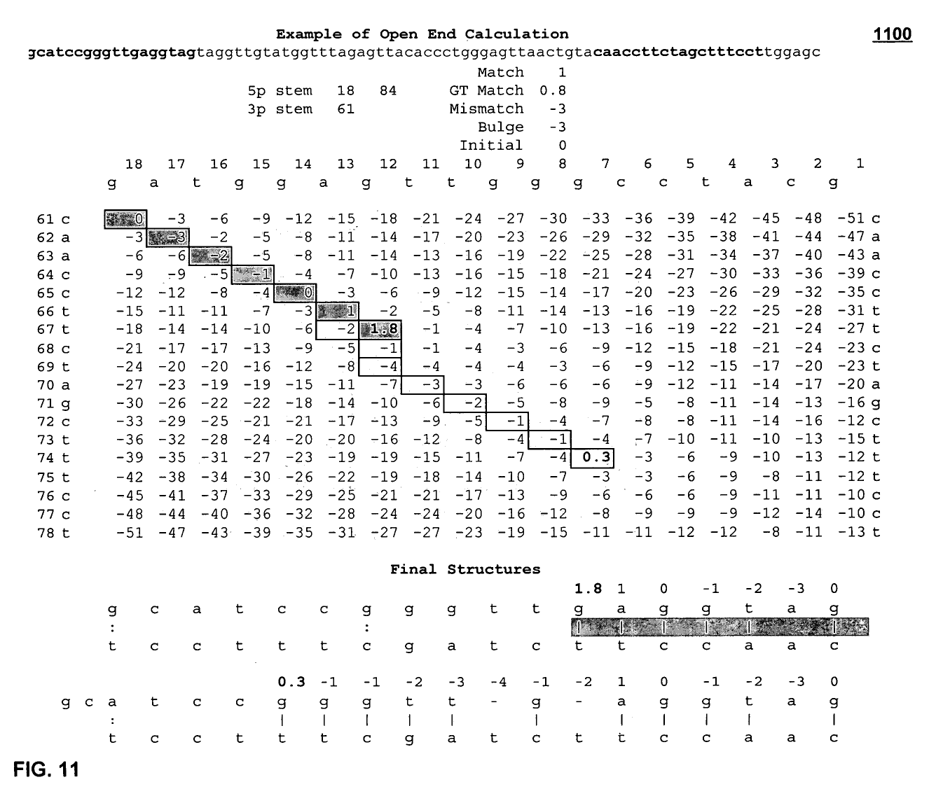

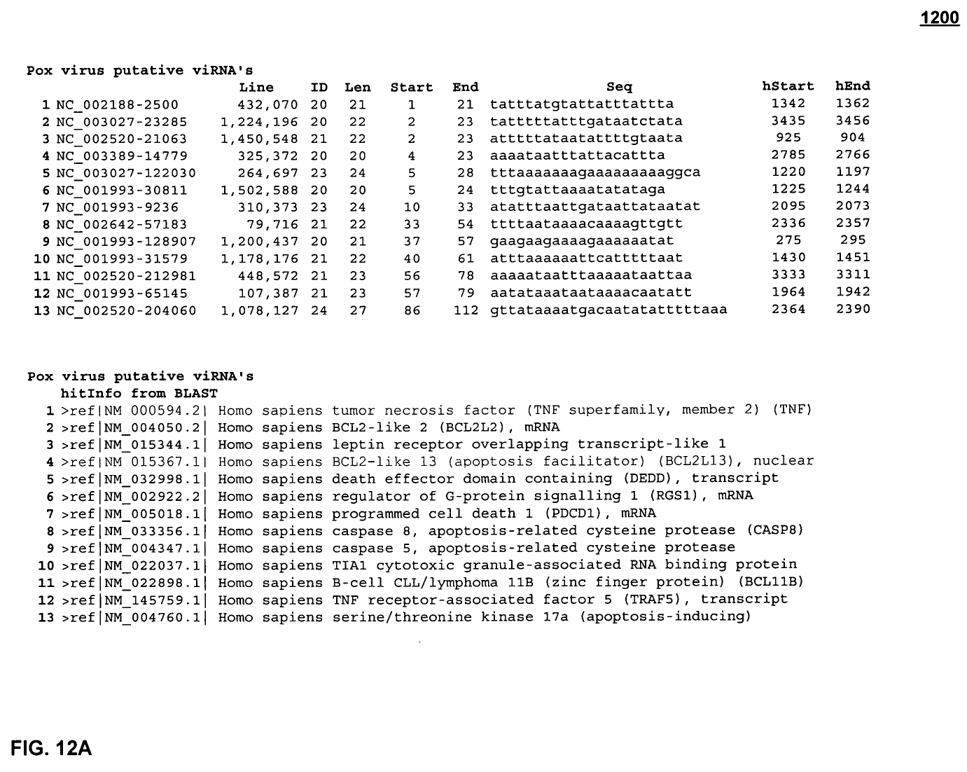

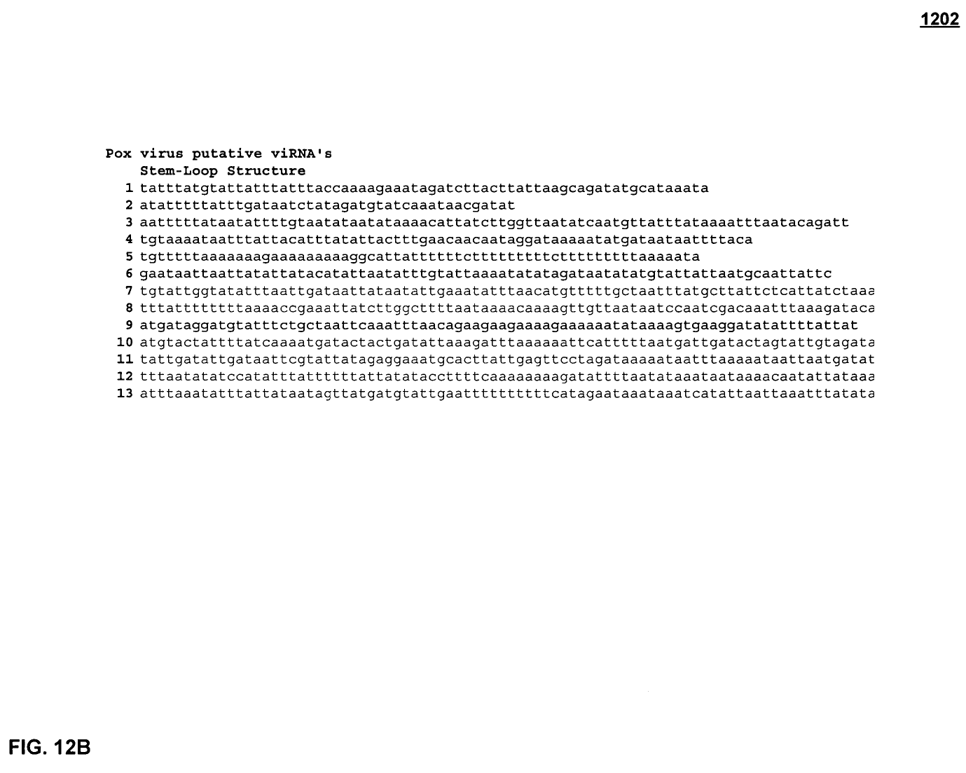

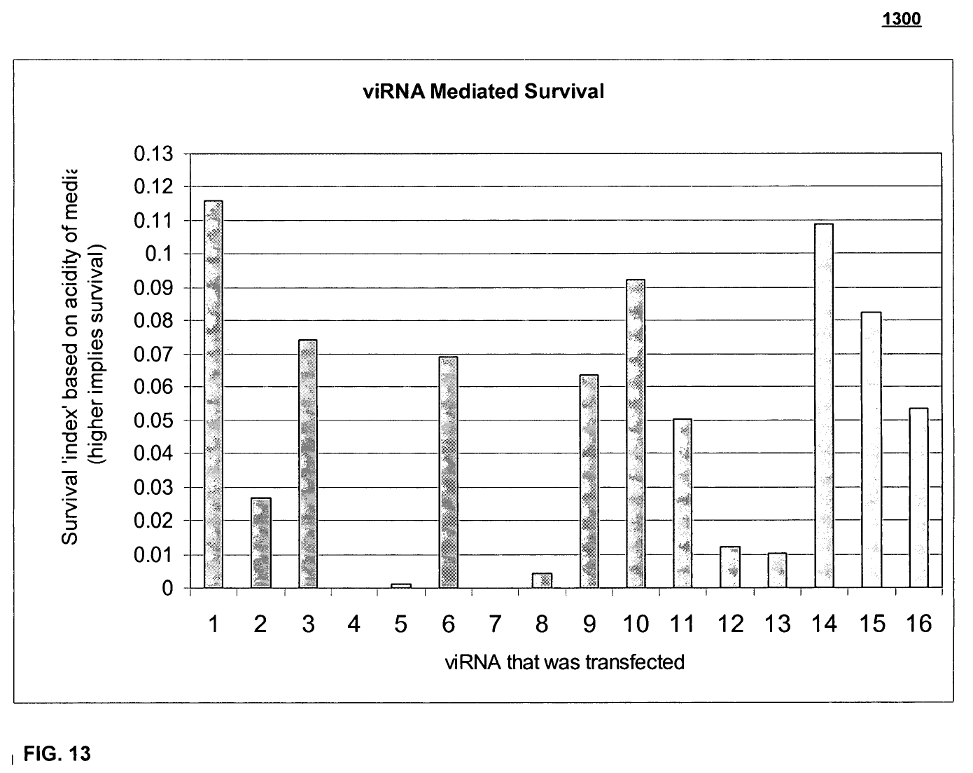

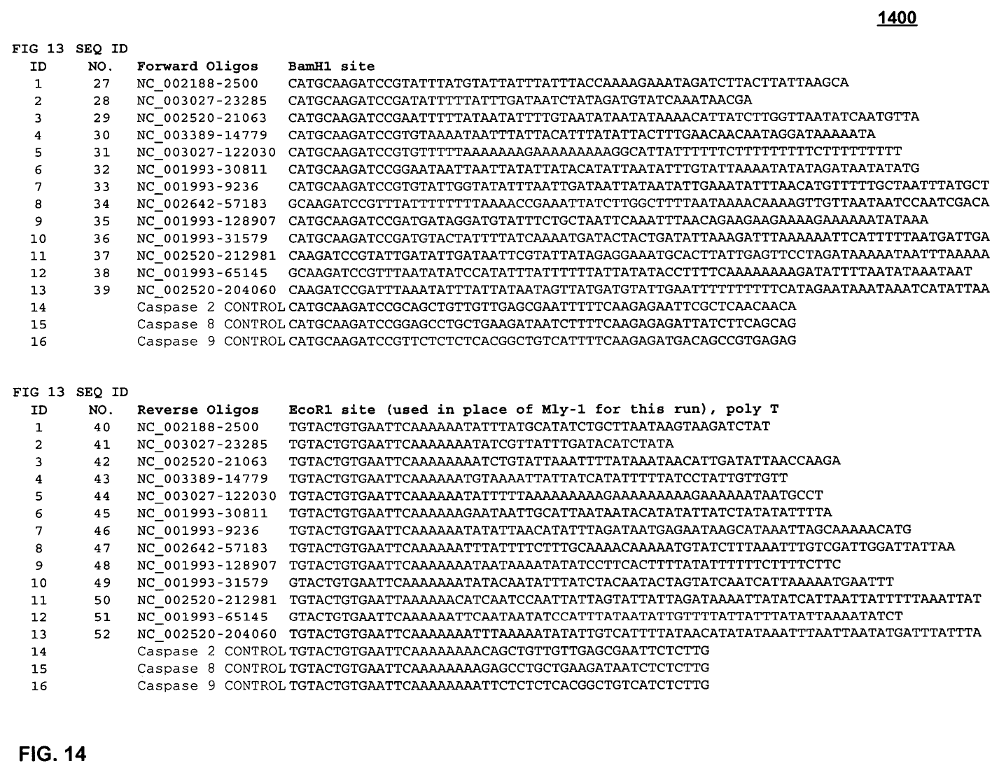

Step 808 shows a fragment of a ranking table. The score is 13.5, the heterogeneity is four, and the conservation is zero for the starred sequence. Step 810 shows one reference of a BLAST output. A 100% match is shown to homo sapiens regulator of G-protein signaling 1 for 20 bases. Step 812 shows one parsing entry of the file from step 810. Step 814 shows PCR off template on a chip and conversion of DNA into an siRNA (viRNA.) Step 815 shows GFP inhibition with 5 or 50 RNAi's in a GFP expressing cell line. Steps 816 and 818 show packaging and amplification. Step 820 shows in vitro transfection. Step 822 shows PCR rescue of protective viRNA's. Step 824 shows a table of the protective function of selected viRNA's. FIG. 9 shows synthesis of viRNA/siRNA molecules via forward and reverse oligos. FIG. 14 shows the forward and reverse oligos for the stem-loop structures of FIG. 12A, FIG. 12B, and FIG. 13. FIG. 10 and FIG. 11 show an example of the loop-end dynamic program table method and the open-end dynamic programming table method respectively.

For the example shown in FIG. 12A, FIG. 12B, FIG. 13, and FIG. 14, the stem-loop viRNA's shown in FIG. 12B were synthesized as forward and reverse oligos shown in FIG. 14 and then screened individually in a cell-based assay. FIG. 12A shows the sequences of viRNA stem-loops that matched homo sapien sequences. FIG. 12B shows the viRNA stem-loop sequences. FIG. 13 shows apoptotic survival index of cells transfected with the viRNA's of FIG. 12B. As an example, 293 cells were transfected in 60 mm tissue culture plates with in vitro transcribed viRNA's using standard liposome transfection techniques. Cells were incubated for 48 hours after transfection, then treated in a standard apoptosis assay which normally induces apoptosis in 100% of 293 cells within 48 hours. A representative article teaching such method is White, E P et al Mol Cell Biol 12:2570-2580, the disclosure of which is incorporated by reference herein. After 96 hours the plates were examined by light microscopy for surviving cells. In addition, the optical density at 560 nm was used to quantify cellular survival (which correlates with lactic acid output of metabolizing (living) cells) based on the color of the growth media. Referring to FIG. 13, controls 14-16 performed as expected and protected the cells from apoptosis. In addition, viRNA's 1, 3, 6, 9, 10, 11, and to a lesser extent viRNA 2, were protective against the apoptotic stimuli. viRNA's 8, 12, and 13 and were within the noise of the assay. FIG. 14 shows the corresponding forward and reverse oligos and the controls for the numbered viRNA's in FIG. 13.

N. Drug and Pharmaceutical Development

In general, a high efficiency cell specific delivery system for in vivo therapeutic use may utilize a number of approaches, including the following: (1) specific delivery through a cultured cell line-specific receptor, (2) delivery of small inhibitory DNA or RNA oligodeoxynucleotides in liposomes with or without specific targeting with monoclonal antibodies directed against specific cell surface receptors; (3) retro viral-mediated transfer of DNA expressing the small inhibitory RNA construct of interest; and (4) direct targeting to cells of oligonucleotides via conjugation to antibodies or other binding proteins that are specific for cell surface receptors that function in a receptor-mediated endocytotic process; (5) specific delivery to cultured cell lines via a replication-defective viral vector.

The viRNA compositions of the invention may be administered as individual therapeutic agents or in combination with other therapeutic agents. They can be administered alone, but are generally administered with a pharmaceutical carrier selected on the basis of the chosen route of administration and standard pharmaceutical practice. The dosage administered will vary depending upon known pharmacokinetic/pharmacodynamic characteristics of the particular agent, and its mode and route of administration, as well as the age, weight, and health (including renal and hepatic function) of the recipient; the nature and extent of disease; kind of concurrent therapy; frequency and duration of treatment; and the effect desired. Usually a daily dose of active ingredient can be about 0.1 to 100 mg per kilogram of body weight. Ordinarily 0.5 to 50, and preferably 1 to 10 mg per kg of body weight per day given in divided doses or in sustained release form (including sustained intravenous infusion) will be effective to achieve the desired effects. Dosage forms suitable for internal administration generally contain about 1 milligram to about 500 milligrams of active ingredient per unit. The active ingredient will ordinarily be present in an amount of about 0.5 to 95% by weight of the total pharmaceutical preparation. It is expected that the small inhibitory DNA or RNA oligonucleotide compositions of the invention may be administered parenterally (e.g., intravenously, preferably by intravenous infusion). For parenteral administration, the compositions will be formulated as a sterile, non-pyrogenic solution, suspension, or emulsion. The preparations may be supplied as a liquid formulation or lyophilized powder to be diluted with a pharmaceutically acceptable sterile, non-pyrogenic parenteral vehicle of suitable tonicity, e.g., water for injection, normal saline, or a suitable sugar-containing vehicle, e.g., D5W, D5/0.45, D5/0.2, or a vehicle containing mannitol, dextrose, or lactose. Suitable pharmaceutical carriers, as well as pharmaceutical necessities for use in pharmaceutical formulations, are described in Remington's Pharmaceutical Sciences, a standard reference text in this field, or the USP/NF.

The present invention provides inhibitory oligonucleotide compounds for use in modulating cellular function, such as apoptosis. Modulation is accomplished by providing pools of different inhibitory oligonucleotide compounds that specifically modulate cellular function, such as apoptosis.

For use in kits and diagnostics, the pools of inhibitory oligonucleotide compounds of the present invention, either alone or in combination with other inhibitory oligonucleotide compounds or therapeutics, can be used as tools in differential and/or combinatorial analyses to elucidate expression patterns of a portion or the entire complement of genes expressed within cells and tissues. Expression patterns within cells or tissues treated with one or more inhibitory oligonucleotide compounds are compared to control cells or tissues not treated with inhibitory oligonucleotide compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds that affect expression patterns. Examples of methods of gene expression analysis known in the art include DNA arrays or microarrays (Brazma and Vilo, FEBS Lett. 480:17-24, 2000; Celis et al., FEBS Lett., 480:2-16, 2000), SAGE (serial analysis of gene expression) (Madden et al., Drug Discov. Today, 5:415-425, 2000), READS (restriction enzyme amplification of digested cDNA's) (Prashar and Weissman, Methods Enzymol., 303:258-72, 1999), TOGA (total gene expression analysis) (Sutcliffe et al., Proc. Natl. Acad. Sci. U.S.A. 97:1976-81, 2000), protein arrays and proteomics (Celis et al., FEBS Lett, 480:2-16, 2000; Jungblut et al., Electrophoresis, 20:2100-10, 2000), expressed sequence tag (EST) sequencing (Celis et al., FEBS Lett, 480:2-16, 2000; Larsson et al., J. Biotechnol, 80:143-57, 2000), subtractive RNA fingerprinting (SuRF) (Fuchs et al., Anal. Biochem., 286:91-98, 2000; Larson et al., Cytometry, 41:203-208, 2000), subtractive cloning, differential display (DD) (Jurecic and Belmont, Curr. Opin. Microbiol, 3:316-21, 2000), comparative genomic hybridization (Carulli et al., J. Cell Biochem. Suppl, 31:286-96, 1998), FISH (fluorescent in situ hybridization) techniques (Going and Gusterson, Eur. J. Cancer 35:1895-904, 1999) and mass spectrometry methods (reviewed in (To, Comb. Chem. High Throughput Scree, 3:235-41, 2000).

A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage. Specific examples of preferred antisense compounds include, for example, oligonucleotides containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone can also be considered to be oligonucleosides. Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkyl-phosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e., a single inverted nucleoside residue that may be a basic (the nucleobase is missing or has a hydroxyl group in place thereof). Various salts, mixed salts but are not limited to, U.S. Pat. Nos. 3,687,808; 4,469,863; 4,476,301; and 5,625,050, and free acid forms are also included.

Representative United States patents that teach the preparation of the above phosphorus-containing linkages include, for example,

| | | | | | |
|---|---|---|---|---|---|
| 5,023,243; | 5,177,196; | 5,188,897; | 5,264,423; | 5,276,019; | |
| 5,278,302; | 5,286,717; | 5,321,131; | 5,399,676; | 5,405,939; | |
| 5,453,496; | 5,455,233; | 5,466,677; | 5,476,925; | 5,519,126; | |
| 5,536,821; | 5,541,306; | 5,550,111; | 5,563,253; | 5,571,799; | |
| 5,587,361; | 5,194,599; | 5,565,555; | 5,527,899; | 5,721,218; | 5,672,697 |

The disclosure of each of which is incorporated by reference herein.

Preferred modified oligonucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; form acetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

Representative United States patents that teach the preparation of the above oligonucleosides: include, but are not limited to, U.S. Pat. Nos. 5,034,506; 5,166,315; 5,185,444 5,264,562 5,235,033;

| | | | | |
|---|---|---|---|---|
| 5,214,134 | 5,216,141 | 5,264,562 | 5,264,564; | 5,405,938; |
| 5,434,257; | 5,466,677; | 5,470,967; | 5,489,677; | 5,541,307; |
| 5,561,225; | 5,596,086; | 5,602,240; | 5,610,289; | 5,602,240; |
| 5,608,046; | 5,610,289; | 5,618,704; | 5,623,070; | 5,663,312; |

| | | | |
|---|---|---|---|
| 5,633,360; | 5,677,437; | 5,792,608; | 5,646,269 and 5,677,439 |

The disclosure of each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is incorporated by reference herein. Further teaching of PNA compounds can be found in Nielsen et al., Science, 254:1497-1500, 1991.

Most preferred embodiments are oligonucleotides with phosphorothioate backbones and oligonucleosides with heteroatom backbones, and in particular —CH2-NH—O—CH2-, —CH2-N (CH3)-O—CH2- (known as a methylene (methylimino) or MMI backbone), —CH2-O—N(CH3)-CH2-, —CH2-N(CH3)-N(CH3)-CH2- and —O—N(CH3)-CH2-CH2- (wherein the native phosphodiester backbone is represented as —O—P—O—CH2-) as described in U.S. Pat. No. 5,489,677, and the amide backbones as described in U.S. Pat. No. 5,602,240. Also preferred are oligonucleotides having morpholino backbone structures as described in U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly preferred are [(CH2)nO]mCH3, O(CH2)nOCH3, O(CH2)nNH2, O(CH2)nCH3, O (CH2)nONH2, and O(CH2)nON [(CH2)nCH3)]2, where n and m are from 1 to about 10. Other preferred oligonucleotides comprise one of the following at the 2′ position: C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2 NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH2CH2OCH3, also known as 2′-O-(2-methoxyethyl) or 2′-MOE) (Martin et al., Helv. Chim. Acta, 78:486-504, 1995) i.e., an alkoxyalkoxy group. A further preferred modification includes 2′-dimethylaminooxyethoxy, i.e., a O═(CH2)2-N—(CH2)2 group, also known as 2′-DMAOE, and 2′-dimethylaminoethoxyethoxy (also known in the art as 2′-β-dimethylaminoethoxyethyl or 2′-DMAEOE), i.e., 2′-O—CH2-O—CH2-N(CH2)2.

A further preferred modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH2-)n, group bridging the 2′ oxygen atom and the 4′ carbon atom wherein n is 1 or 2. LNAs and preparation thereof are described in WO 98/39352 and WO 99/14226. Other preferred modifications include 2′-methoxy (2′-O—CH2), 2′-aminopropoxy (2′-OCH2-CH2CH2-NH2), 2′-alkyl (2′-CH2-CH═CH2), 2′-O-alkyl (2′-O—CH2-CH═CH2) and 2′-fluoro (2′-F). The 2′modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957;

| | | | | |
|---|---|---|---|---|
| 5,118,800; | 5,319,080; | 5,359,044; | 5,393,878; | 5,446,137; |
| 5,466,786; | 5,514,785; | 5,519,134; | 5,567,811; | 5,576,427; |
| 5,591,722; | 5,597,909; | 5,610,300; | 5,627,053; | 5,639,873; |

U.S. Pat. Nos. 5,646,265; 5,658,873; 5,670,633; 5,792,747; and 5,700,920, the disclosure of each of which is incorporated by reference herein.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C°C—CH2) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2 (3H)-one), phenothiazine cytidine (1H-pyrimido[5,4-b][1,4]benzothiazin-2 (3H)-one), G-clamps such as a substituted phenoxazine cytidine (e.g. 9-(2-aminoethoxy)-H-pyrimido[5,4-b][1,4]benzoxazin-2 (3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science and Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications. Representative United States patents that teach the preparation of certain of the above noted modified nucleobases as well as other modified nucleobases include, but are not limited to U.S. Pat. No. 3,687,808, as well as U.S. Pat. Nos.:

| | | | | |
|---|---|---|---|---|
| 4,845,205; | 5,130,302; | 5,134,066; | 5,175,273; | 5,367,066; |
| 5,432,272; | 5,457,187; | 5,459,255; | 5,484,908; | 5,502,177; |
| 5,525,711; | 5,552,540; | 5,587,469; | 5,594,121, | 5,596,091; |
| 5,614,617; | 5,645,985; | 5,830,653; | 5,763,588; | 6,005,096; |

5,681,941, and 5,750,692 the disclosure of each of which is incorporated by reference herein.

Another modification of the oligonucleotides of the invention involves chemically linking to the oligonucleotide one or more moieties or conjugates that enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties include groups that improve oligomer uptake, distribution, metabolism or excretion. Conjugate moieties include but are not limited to lipid moieties such as a cholesterol moiety (Letsinger et al., Proc. Natl. Acad. Sci. USA, 86:6553-6556, 1989), cholic acid (Manoharan et al., Bioorg. Med. Chem. Let., 4:1053-1060, 1994), a thioether, e.g., hexyl-S-tritylthiol (Manoharan et al., Ann. N.Y. Acad. Sci., 660:306-309, 1992; Manoharan et al., Bioorg. Med. Chem. Let., 3:2765-2770, 1993), a thiocholesterol (Oberhauser et al., Nucl. Acids Res., 20: 533-538, 1992), an aliphatic chain, e.g., dodecandiol or undecyl residues (Saison-Behmoaras et al., EMBO J., 10:1111-1118, 1991; Kabanov et al., FEBS Lett., 259:327-330, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-3654, 1995; Shea et al., Nucl. Acids Res., 18:3777-3783, 1990), a polyamine or a polyethylene glycol chain (Manoharan et al., Nucleosides & Nucleotides, 14:969-973, 1995), or adamantane acetic acid (Manoharan et al., Tetrahedron Lett., 36:3651-3654, 1995), a palmityl moiety (Mishra et al., Biochim. Biophys. Acta, 1264:229-237, 1995), or an octadecylamine or hexylamino-carbonyl-oxycholesterol moiety (Crooke et al., J. Pharmacol. Exp. Ther., 277:923-937, 1996). Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, the disclosure of each of which is incorporated by reference herein.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide-increased resistance to nuclease degradation, increased cellular uptake, and/or increased binding affinity for the target nucleic acid. An additional region of the oligonucleotide may serve as a substrate for enzymes capable of cleaving RNA:RNA or RNA:RNA hybrids. By way of example, RNA'se H is a cellular endonuclease, which cleaves the RNA strand of an RNA:DNA duplex. Activation of RNAase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art. Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos.

| | | | | |
|---|---|---|---|---|
| 5,013,830; | 5,149,797; | 5,220,007; | 5,256,775; | 5,366,878; |
| 5,403,711; | 5,491,133; | 5,565,350; | 5,623,065; | 5,652,355; |

5,652,356; and 5,700,922, the disclosure of each of which is incorporated by reference herein.

The antisense compounds used in accordance with this invention may be conveniently and routinely made through the technique of solid phase synthesis. For example, equipment for such synthesis is sold by several vendors including, Applied Biosystems of Foster City, Calif. Any other means for such synthesis known in the art may additionally or alternatively be employed. Similar techniques to prepare oligonucleotides are the phosphorothioates and alkylated derivatives.

The antisense compounds of the invention are synthesized in vitro and do not include antisense compositions of biological origin, or genetic vector constructs designed to direct the in vivo synthesis of antisense molecules. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127;

| | | | | |
|---|---|---|---|---|
| 5,521,291; | 5,543,158; | 5,547,932; | 5,583,020; | 5,591,721; |
| 4,426,330; | 4,534,899; | 5,013,556; | 5,108,921; | 5,213,804; |
| 5,227,170; | 5,264,221; | 5,356,633; | 5,395,619; | 5,416,016; |
| 5,417,978; | 5,462,854; | 5,469,854; | 5,512,295; | 5,527,528; |

| | | | |
|---|---|---|---|
| 5,534,259; | 5,543,152; | 5,556,948; | 5,580,575; and 5,595,756, |

The disclosure of each of which is incorporated by reference herein.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents. The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 or in WO 94/26764 and U.S. Pat. No. 5,770,713.

The term “pharmaceutically acceptable salts” refers to physiologically and pharmaceutically acceptable salts of compounds of the invention: i.e., salts that retain the desired biological activity of the parent compound and do not impart undesired toxicological effects thereto. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 66:1-19, 1977). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example, glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations include alkaline, alkaline earth, ammonium and quaternary ammonium cations. Carbonates or hydrogen carbonates are also possible.

For oligonucleotides, preferred examples of pharmaceutically acceptable salts include, but are not limited to, (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The oligonucleotide compounds of the present invention can be utilized for diagnostics, therapeutics, prophylaxis and as research reagents and kits. For therapeutics, an animal, preferably a human, suspected of having a disease or disorder which can be treated by administering oligonucleotide or antisense compounds in accordance with this invention. The compounds of the invention can be utilized in pharmaceutical compositions by adding an effective amount of an oligonucleotide or antisense compound to a suitable pharmaceutically acceptable diluent or carrier. Use of the oligonucleotide compounds and methods of the invention may also be useful prophylactically, e.g., to prevent or delay a condition or infection.

The oligonucleotide compounds of the invention are useful for research and diagnostics, because these compounds hybridize to nucleic acids, enabling sandwich and other assays to easily be constructed. Hybridization of the oligonucleotide compounds of the invention with a nucleic acid can be detected by means known in the art. Such means may include conjugation of an enzyme to the oligonucleotide, radiolabeling of the oligonucleotide or any other suitable detection means. Kits using such detection means may also be prepared.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. siRNA compounds with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). Oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-10 alkyl ester (e.g., isopropylmyristate IPM), monoglyceride, diglyceride or pharmaceutically acceptable salt thereof.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. siRNA compounds of the invention may be delivered orally in granular form including sprayed dried particles, or complexed to form micro or nanoparticles. Oligonucleotide complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyomithine, polyspermines, protamine, polyvinylpyridine, polythiodiethylamino-methylethylene P (TDAE), polyaminostyrene e.g., p-amino), poly (methylcyanoacrylate), poly (ethylcyanoacrylate), poly (butylcyanoacrylate), poly (isobutylcyanoacrylate), poly (isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. The pharmaceutical formulations of the present invention, which may be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

Emulsions

The pharmaceutical compositions of the present invention may be prepared and formulated as emulsions. Emulsions are typically heterogenous systems of one liquid dispersed in another in the form of droplets usually exceeding 0.1 μm in diameter. (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug that may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199). Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, non-swelling clays such as bentonite, attapulgite, hectorite, kaolin, montmorillonite, colloidal aluminum silicate and colloidal magnesium aluminum silicate, pigments and non-polar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations, via dermatological, oral and parenteral routes, and methods for their manufacture has been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of oligonucleotide compounds are formulated as microemulsions. A microemulsion may be defined as a system of water, oil and amphiphile that is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been studied and has yielded a comprehensive knowledge how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously. Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML3 10), tetraglycerol monooleate (M03 10), hexaglycerol monooleate (P0310), hexaglycerol pentaoleate (P0500), decaglycerol monocaprate (MCA750), decaglycerol monooleate (M0750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 11:1385-1390, 1994; Ritschel, Meth. Find. Exp. Clin. Pharmacol., 13:205, 1993). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 11:1385, 1994; Ho et al., J. Pharm. Sci., 85:138-143, 1996). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories: surfactants, fatty acids, bile salts, chelating agents, and non-chelating non-surfactants (Lee et al., Critical Reviews in Therapeutic Drug Carrier Systems, p. 92, 1991

Liposomes

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross-intact mammalian skin, lipid vesicles must pass through a series of fine pores, each with a diameter less than 50 nm, under the influence of a suitable transdermal gradient. Therefore, it is desirable to use a liposome that is highly deformable and able to pass through such fine pores. Advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNA's have been administered to the skin. The majority of applications resulted in the targeting of the upper epidermis.

Liposomes fall into two broad classes. Cationic liposomes are positively charged liposomes that interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 147:980-985, 1987). Liposomes that are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., J. Controlled Release, 19:269-274, 1992).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g., as a solution or as an emulsion) was ineffective (Weiner et al., J. Drug Targeting, 2:405-410, 1992). Further, an additional study tested the efficacy of interferon administered as part of a liposomal formulation to the administration of interferon using an aqueous system, and concluded that the liposomal formulation was superior to aqueous administration (du Plessis et al., Anti Human Papillomavirus (HPV) Viral Research, 18:259-265, 1992).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al., S.T.P. Pharma. Sci., 4:6, 466, 1994). Liposomes also include “sterically stabilized” liposomes, a term that refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside GM!, or (B) is derivatized with one or more hydrophilic polymers, such as a polyethylene glycol (PEG) moiety. While not wishing to be bound by any particular theory, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 223:42, 1987; Wu et al., Cancer Research, 53:3765, 1993).

Various liposomes can comprise one or more glycolipids. Papahadjopoulos et al. (Ann. N.Y. Acad. Sci., 507:64, 1987) reported the ability of monosialoganglioside GM1, galactocerebroside sulfate and phosphatidylinositol to improve blood half-lives of liposomes. These findings were expounded upon by Gabizon et al. (Proc. Natl. Acad. Sci. U.S.A., 85:6949, 1988). U.S. Pat. No. 4,837,028 and WO 88/04924, disclose liposomes comprising (1) sphingomyelin and (2) the ganglioside GM! or a galactocerebroside sulfate ester. U.S. Pat. No. 5,543,152 discloses liposomes comprising sphingomyelin. Liposomes comprising 1,2-sn-dimyristoylphosphatidylcholine are disclosed in WO 97/13499.

Many liposomes comprise lipids derivatized with one or more hydrophilic polymers. Sunamoto et al. (Bull. Chem. Soc. Jpn., 53:2778, 1980) described liposomes comprising a nonionic detergent, 2C1215G, which contains a PEG moiety. Illum et al. (FEBS Lett. 167:79, 1984) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described in U.S. Pat. Nos. 4,426,330 and 4,534,899. Klibanov et al. (FEBS Lett. 268:235, 1990) described experiments demonstrating that liposomes comprising phosphatidylethanolamine (PE) derivatized with PEG or PEG stearate have significant increases in blood circulation half-lives. Blume et al. (Biochimica et Biophysica Acta 1029:91, 1990) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent EP 0 445 131 B1 and WO 90/04384. Liposome compositions containing 1-20 mole percent of PE derivatized with PEG are described in U.S. Pat. Nos. 5,013,556 and 5,356,633 and in U.S. Pat. No. 5,213,804 and European Patent EP 0 496 813 B1. Liposomes comprising a number of other lipid-polymer conjugates are disclosed in WO 91/05545 and U.S. Pat. No. 5,225,212 and in WO 94/20073. Liposomes comprising PEG-modified ceramide lipids are described in WO 96/10391. U.S. Pat. Nos. 5,540,935 and 5,556,948 describe PEG-containing liposomes that can be further derivatized with functional moieties on their surfaces. WO 96/40062 discloses methods for encapsulating high molecular weight nucleic acids in liposomes. U.S. Pat. No. 5,264,221 discloses protein-bonded liposomes and asserts that the contents of such liposomes may include an antisense RNA. U.S. Pat. No. 5,665,710 describes certain methods of encapsulating oligodeoxynucleotides in liposomes. WO 97/04787 discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets, which are so highly deformable that they are easily able to penetrate through pores, which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class. If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps. If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class. If the surfactant molecule has the ability to carry either a positive or negative charge, the surfactant is classified as amphoteric. Amphoteric surfactants include acrylic acid derivatives, substituted alkylamides, N-alkylbetaines and phosphatides. The use of surfactants in drug products, formulations and in emulsions has been reviewed (Rieger, in Pharmaceutical Dosage Forms, Marcel Dekker, Inc., New York, N.Y., 1988, p. 285).

Penetration Enhancers

In one embodiment, the present invention employs various penetration enhancers to affect the efficient delivery of nucleic acids, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs. Penetration enhancers may be classified as belonging to one of five broad categories: surfactants, fatty acids, bile salts, chelating agents, and non-chelating non-surfactants (Lee et al., Critical Reviews in Therapeutic Drug Carrier Systems, 1991, p. 92).

Surfactants

Surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p. 92); and perfluorochemical emulsions, such as FC-43. Takahashi et al., J. Pharm. Pharmacol. 40:252, 1988).

Fatty Acids

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1monocaprate, 1-dodecylazacycloheptan-2-one, acylcamitines, acylcholines, C1-10, alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, p. 92, 1991; Muranishi, Critical Reviews in Therapeutic Drug Carrier Systems, 7:1-33, 1990; El Hariri et al., J. Pharm. Pharmacol., 44:651-654, 1992).

Bile Salts

The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro 24,25-dihydro-fusidate (STDHF), sodium glycodihydrofusidate and polyoxyethylene-9-lauryl ether (POE) (Lee et al., Critical Reviews in Therapeutic Drug Carrier Systems, page 92, 1991; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 7:1-33, 1990; Yamamoto et al., J. Pharm. Exp. Ther., 263:25, 1992; Yamashita et al., J. Pharm. Sci., 79:579-583, 1990).

Chelating Agents

Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNA'se inhibitors, as most characterized DNA nucleases require a divalent metal for catalysis and are thus inhibited by chelating agents (Jarrett, J. Chromatogr., 618:315-339, 1993). Chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., Critical Reviews in Therapeutic Drug Carrier Systems, 1991, page 92; Muranishi, Critical Reviews in Therapeutic Drug Carrier Systems, 7:1-33, 1990; Buur et al., J. Control Rel., 14:43-51, 1990).

Non-chelating non-surfactants penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 7:1-33, 1990). This class of penetration enhancers includes, for example, unsaturated cyclic ureas, 1-alkyl- and 1alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol, 39:621-626, 1987).

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), each enhance the cellular uptake of oligonucleotides. Other agents may be utilized to enhance the penetration of the administered nucleic acids, including glycols such as ethylene glycol and propylene glycol, pyrrols such as 2-pyrrol, azones, and terpenes such as limonene and menthone.

Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The co-administration of a nucleic acid and a carrier compound, typically with an excess of the latter substance, can result in a substantial reduction of the amount of nucleic acid recovered in the liver, kidney or other extra-circulatory reservoirs, presumably due to competition between the carrier compound and the nucleic acid for a common receptor. For example, the recovery of a partially phosphorothioate oligonucleotide in hepatic tissue can be reduced when it is co-administered with polyinosinic acid, dextran sulfate, polycytidic acid or 4-acetamido-4′isothiocyanostilbene-2,2′-disulfonic acid (Miyao et al., Antisense Res. Dev., 5:115-121, 1995; Takakura et al., Antisense & Nucl. Acid Drug Dev., 6:177-183, 1996).

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.). Pharmaceutically acceptable organic or inorganic excipient suitable for non-parenteral administration that do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of oligonucleotides may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration that do not deleteriously react with nucleic acids can be used. Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components

The compositions of the present invention may additionally contain other adjunct components conventionally found in pharmaceutical compositions, at their art-established usage levels. For example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation. Aqueous suspensions may contain substances that increase the viscosity of the suspension including, for example, sodium carboxymethylcellulose, sorbitol and/or dextran. The suspension may also contain stabilizers.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can determine optimum dosages, dosing methodologies and repetition rates. Optimum dosages may vary depending on the relative potency of individual oligonucleotides, and can be estimated based on EC50s found to be effective in in vitro and in vivo animal models. In general, dosage is from 0.01 mg to 100 mg per kg of body weight, and may be given once or more daily, weekly, monthly or yearly, or even once every 2 to 20 years. Persons of ordinary skill in the art can estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 mg to 100 mg per kg of body weight, once or more daily, to once every 20 years.

We claim:

1. A method in a data processing system for identifying candidate interfering stem-loop sequences from a candidate genome of a target organism for use in treating a condition, comprising:
(a) reading a sequence of the candidate genome from a computer readable medium;
(b) identifying a first window having a defined length of sequential bases along the sequence and subsequent windows having the defined length, wherein each subsequent window is overlapping a preceding window along the sequence;
(c) finding an optimum base pairing for each window, wherein the optimum base pairing is determined by calculating a stem-loop quality numeric determination using a dynamic programming method, wherein the dynamic programming method comprises a loop-end method or a base island method; and
(d) reporting each stem-loop quality numeric determination and the sequential bases corresponding thereto of the optimum base pairing from the dynamic programming method to identify the candidate interfering stem-loop sequences.

2. The method of claim 1, wherein the defined length of each window comprises from about 10 bases of the sequence to about 200 bases of the sequence.

3. The method of claim 1, wherein the dynamic programming method comprises the loop-end method, and wherein the loop-end method comprises:
(a) creating a two-dimensional dynamic programming table for each window to fit the sequential bases of each window along a horizontal top of the two-dimensional dynamic programming table and to fit the sequential bases of each window along a vertical left side of the two-dimensional dynamic programming table;
(b) representing the sequential bases of each window along the horizontal top of the two-dimensional dynamic programming table, forming a horizontal base top;
(c) representing the sequential bases of each window from the opposite direction along the vertical left side starting at the horizontal top of the two-dimensional dynamic programming table, forming a vertical base side;
(d) calculating a table quality score for entry into each cell of a top-left half of the two-dimensional dynamic programming table corresponding to each base-base interaction between the horizontal base top and the vertical base side using a scoring method, comprising (i) adding a match number to an initial quality score for each A-U, U-A, C-G, or G-C base match, forming a cumulative score, (ii) adding a partial-match number to the cumulative score for each G-U or U-G base match, (iii) adding a five-bulge number to the cumulative score for each 5 prime side bulge, (iv) adding a three-bulge number to the cumulative score for each 3 prime side bulge, and (v) adding a mismatch number to the cumulative score for each A-A, C-C, G-G, U-U, A-C, C-A, A-G, G-A, C-U, or U-C mismatch;
(e) locating a highest value of each table quality score corresponding to the optimum base pairing for each window; and
(f) storing the highest value and corresponding base sequence of each window when the highest value exceeds a threshold value, a stem length exceeds a minimum stem length, and a loop size is greater than a minimum loop size.

4. The method of claim 3, wherein the initial quality score is approximately zero, the match number is from about 0.5 to about 3.0, the partial match number is from about 0.25 to about 1.5, five prime bulge number is from about −0.5 to about −6.0, the three prime bulge number is from about −0.5 to about −6.0, and the mismatch number is from about −0.5 to about −6.0.

5. The method of claim 3, wherein the threshold value is from about 5 to about 15, the minimum stem length is from about 5 to about 25 base pairs, and the minimum loop size is from about 3 to about 10 bases.

6. The method of claim 1, wherein the dynamic programming method comprises the base island method, and wherein the base island method comprises:
(a) pairing bases by folding in half each window to match bases from each half having an unmatched base at a loop end forming a point folded window;
(b) pairing bases by folding in half each window to match bases from each half having matched bases at a loop end forming a blunt folded window;
(c) identifying a base pair island for each folded window by searching each folded window for a consecutively bound base pairing grouping until a loop size range is exceeded; and
(d) finding an optimum base sequence pairing for each window on both sides of the base pair island by summing a loop-end quality and an open-end quality, wherein the qualities are calculated by (i) calculating the loop-end quality in a loop-end region of the consecutively bound base pair grouping using the loop-end method and (ii) calculating the open-end quality in an open-end region of the consecutively bound base pair grouping using an open-end method.

7. The method of claim 6, wherein the consecutively bound base pairing grouping is from about 3 to about 8 base pairs, and the loop size range is from about 3 to about 70 bases.

8. The method of claim 6, wherein the loop-end method comprises:
(a) creating a two-dimensional dynamic programming table for each window to fit the sequential bases of each window along a horizontal top of the two-dimensional dynamic programming table and to fit the sequential bases of each window along a vertical left side of the two-dimensional dynamic programming table;
(b) representing the sequential bases of each window along the horizontal top of the two-dimensional dynamic programming table, forming a horizontal base top;
(c) representing the sequential bases of each window from the opposite direction along the vertical left side starting at the horizontal top of the two-dimensional dynamic programming table, forming a vertical base side;
(d) calculating a table quality score for entry into each cell of a top-left half of the two-dimensional dynamic programming table corresponding to each base-base interaction between the horizontal base top and the vertical base side using a scoring method, comprising (i) adding a match number to an initial quality score for each A-U, U-A, C-G, or G-C base match, forming a cumulative score, (ii) adding a partial-match number to the cumulative score for each G-U or U-G base match, (iii) adding a five-bulge number to the cumulative score for each 5 prime side bulge, (iv) adding a three-bulge number to the cumulative score for each 3 prime side bulge, and (v) adding a mismatch number to the cumulative score for each A-A, C-C, G-G, U-U, A-C, C-A, A-G, G-A, C-U, or U-C mismatch;
(e) locating a highest value of each table quality score corresponding to the optimum base pairing for each window; and
(f) storing the highest value and corresponding base sequence of each window when the highest value exceeds a threshold value, a stem length exceeds a minimum stem length, and a loop size is greater than a minimum loop size.

9. The method of claim 8, wherein the initial quality score is approximately zero, the match number is from about 0.5 to about 3.0, the partial match number is from about 0.25 to about 1.5, five prime bulge number is from about −0.5 to about −6.0, the three prime bulge number is from about −0.5 to about −6.0, and the mismatch number is from about −0.5 to about −6.0.

10. The method of claim 8, wherein the threshold value is from about 5 to about 15, the minimum stem length is from about 5 to about 25 base pairs, and the minimum loop size is from about 3 to about 10 bases.

11. The method of claim 6, wherein the open-end method comprises:
(a) creating a two-dimensional dynamic programming table for each window to fit the sequential bases of each window along a horizontal top of the two-dimensional dynamic programming table and to fit the sequential bases of each window along a vertical left side of the two-dimensional dynamic programming table;
(b) representing the sequential bases of each window along the horizontal top of the two-dimensional dynamic programming table, forming a horizontal base top;
(c) representing the sequential bases of each window from the opposite direction along the vertical left side starting at the horizontal top of the two-dimensional dynamic programming table, forming a vertical base side;
(d) calculating a table quality score for entry into each cell of the two-dimensional dynamic programming table corresponding to each base-base interaction between the horizontal base top and the vertical base side using a scoring method, comprising (i) adding a match number to an initial quality score for each A-U, U-A, C-G, or G-C base match, forming a cumulative score, (ii) adding a partial-match number to the cumulative score for each G-U or U-G base match, (iii) adding a five-bulge number to the cumulative score for each 5 prime side bulge, (iv) adding a three-bulge number to the cumulative score for each 3 prime side bulge, and (v) adding a mismatch number to the cumulative score for each A-A, C-C, G-G, U-U, A-C, C-A, A-G, G-A, C-U, or U-C mismatch;
(e) locating a highest value of each table quality score corresponding to the optimum base pairing for each window; and
(f) storing the highest value and corresponding base sequence of each window when the highest value exceeds a threshold value, a stem length exceeds a minimum stem length, and a loop size is greater than a minimum loop size.

12. The method of claim 11, Wherein the initial quality score is approximately zero, the match number is from about 0.5 to about 3.0, the partial match number is from about 0.25 to about 1.5, five prime bulge number is from about −0.5 to about −6.0, the three prime bulge number is from about −0.5 to about −6.0, and the mismatch number is from about −0.5 to about −6.0.

13. The method of claim 11, wherein the threshold value is from about 5 to about 15, the minimum stem length is from about 5 to about 25 base pairs, and the minimum loop size is from about 3 to about 10 bases.

14. The method of claim 1, wherein the candidate genome comprises at least two strains of a viral genome.

15. The method of claim 14, wherein the viral genome is a pox viral genome.

16. The method of claim 1, wherein the candidate genome comprises a sequenced genome.

17. The method of claim 1, wherein the viral genome is a sequence obtained from a viral family, wherein the viral family is selected from the group consisting of:
“CrPV-like viruses”, “HEV-like viruses”, “SNDV-like viruses”, Adenoviridae, Allexivirus, Arenaviridae, Arteriviridae, Ascoviridae, Asfarviridae, Astroviridae, Baculoviridae, Barnaviridae, Benyvirus, Bimaviridae, Bomaviridae, Bromoviridae, Bunyaviridae, Caliciviridae, Capillovirus, Carlavirus, Caulimoviridae, Circoviridae, Closteroviridae, Comoviridae, Coronaviridae, Corticoviridae, Cystoviridae, Deltavirus, Filoviridae, Flaviviridae, Foveavirus, Furovirus, Fuselloviridae, Geminiviridae, Hepadnaviridae, Herpesviridae, Hordeivirus, Hypoviridae, Idaeovirus, Inoviridae, Iridoviridae, Leviviridae, Lipothrixviridae, Luteoviridae, Marafivirus, Metaviridae, Microviridae, Myoviridae, Nanovirus, Namaviridae, Nodaviridae, Ophiovirus, Orthomyxoviridae, Ourmiavirus, Papillomaviridae, Paramyxoviridae, Partitiviridae, Parvoviridae, Pecluvirus, Phycodnaviridae, Picornaviridae, Plasmaviridae, Podoviridae, Polydnaviridae, Polyomaviridae, Pomovirus, Potexvirus, Potyviridae, Poxviridae, Pseudoviridae, Reoviridae, Retroviridae, Rhabdoviridae, Rhizidiovirus, Rudiviridae, Sequiviridae, Siphoviridae, Sobemovirus, Tectiviridae, Tenuivirus, Tetraviridae, Tobamovirus, Tobravirus, Togaviridae, Tombusviridae, Totiviridae, Trichovirus, Tymovirus, Umbravirus, Varicosavirus, and Vitivirus.

18. A method for identifying interfering stem-loop sequences from a candidate genome for use in treatment of a condition in a target organism, comprising:
(a) selecting the candidate genome and the target organism; and
(b) identifying the interfering stem-loop sequences from the candidate genome using a data processing system by (i) reading a sequence of the candidate genome from a computer readable medium, (ii) identifying a first window having a defined length of sequential bases along the sequence and subsequent windows having the defined length, wherein each subsequent window is overlapping a preceding window along the sequence, (iii) finding an optimum base pairing for each window, wherein the optimum base pairing is determined by calculating a stem-loop quality numeric determination using a dynamic programming method, wherein the dynamic programming method comprises a loop-end method or a base island method, and (iv) reporting each stem-loop quality numeric determination and the sequential bases corresponding thereto of the optimum base pairing from the dynamic programming method to identify the candidate interfering stem-loop sequences.

19. A method, according to claim 18, wherein the defined length of each window comprises from about 10 bases of the sequence to about 200 bases of the sequence.

20. A method, according to claim 18, wherein the dynamic programming method comprises the loop-end method, and wherein the loop-end method comprises:
(a) creating a two-dimensional dynamic programming table for each window to fit the sequential bases of each window along a horizontal top of the two-dimensional dynamic programming table and to fit the sequential bases of each window along a vertical left side of the two-dimensional dynamic programming table;
(b) representing the sequential bases of each window along the horizontal top of the two-dimensional dynamic programming table, forming a horizontal base top;
(c) representing the sequential bases of each window from the opposite direction along the vertical left side starting at the horizontal top of the two-dimensional dynamic programming table, forming a vertical base side;
(d) calculating a table quality score for entry into each cell of a top-left half of the two-dimensional dynamic programming table corresponding to each base-base interaction between the horizontal base top and the vertical base side using a scoring method, comprising (i) adding a match number to an initial quality score for each A-U, U-A, C-G, or G-C base match, forming a cumulative score, (ii) adding a partial-match number to the cumulative score for each G-U or U-G base match, (iii) adding a five-bulge number to the cumulative score for each 5 prime side bulge, (iv) adding a three-bulge number to the cumulative score for each 3 prime side bulge, and (v) adding a mismatch number to the cumulative score for each A-A, C-C, G-G, U-U, A-C, C-A, A-G, G-A, C-U, or U-C mismatch;
(e) locating a highest value of each table quality score corresponding to the optimum base pairing for each window; and
(f) storing the highest value and corresponding base sequence of each window when the highest value exceeds a threshold value, a stem length exceeds a minimum stem length, and a loop size is greater than a minimum loop size.

21. The method of claim 20, wherein the initial quality score is approximately zero, the match number is from about 0.5 to about 3.0, the partial match number is from about 0.25 to about 1.5, five prime bulge number is from about −0.5 to about −6.0, the three prime bulge number is from about −0.5 to about −6.0, and the mismatch number is from about −0.5 to about −6.0.

22. The method of claim 20, wherein the threshold value is from about 5 to about 15, the minimum stem length is from about 5 to about 25 base pairs, and the minimum loop size is from about 3 to about 10 bases.

23. The method of claim 18, wherein the dynamic programming method comprises the base island method, and wherein the base island method comprises:
(a) pairing bases by folding in half each window to match bases from each half having an unmatched base at a loop end forming a point folded window;
(b) pairing bases by folding in half each window to match bases from each half having matched bases at a loop end forming a blunt folded window;
(c) identifying a base pair island for each folded window by searching each folded window for a consecutively bound base pairing grouping until a loop size range is exceeded; and
(d) finding an optimum base sequence pairing for each window on both sides of the base pair island by summing a loop-end quality and an open-end quality, wherein the qualities are calculated by (i) calculating the loop-end quality in a loop-end region of the consecutively bound base pair grouping using the loop-end method and (ii) calculating the open-end quality in an open-end region of the consecutively bound base pair grouping using an open-end method.

24. The method of claim 23, wherein the consecutively bound base pairing grouping is from about 3 to about 8 base pairs, and the loop size range is from about 3 to about 70 bases.

25. The method of claim 23, wherein the loop-end method comprises:
(a) creating a two-dimensional dynamic programming table for each window to fit the sequential bases of each window along a horizontal top of the two-dimensional dynamic programming table and to fit the sequential bases of each window along a vertical left side of the two-dimensional dynamic programming table;
(b) representing the sequential bases of each window along the horizontal top of the two-dimensional dynamic programming table, forming a horizontal base top;
(c) representing the sequential bases of each window from the opposite direction along the vertical left side starting at the horizontal top of the two-dimensional dynamic programming table, forming a vertical base side;
(d) calculating a table quality score for entry into each cell of a top-left half of the two-dimensional dynamic programming table corresponding to each base-base interaction between the horizontal base top and the vertical base side using a scoring method, comprising (i) adding a match number to an initial quality score for each A-U, U-A, C-G, or G-C base match, forming a cumulative score, (ii) adding a partial-match number to the cumulative score for each G-U or U-G base match, (iii) adding a five-bulge number to the cumulative score for each 5 prime side bulge, (iv) adding a three-bulge number to the cumulative score for each 3 prime side bulge, and (v) adding a mismatch number to the cumulative score for each A-A, C-C, G-G, U-U, A-C, C-A, A-G, G-A, C-U, or U-C mismatch;
(e) locating a highest value of each table quality score corresponding to the optimum base pairing for each window; and
(f) storing the highest value and corresponding base sequence of each window when the highest value exceeds a threshold value, a stem length exceeds a minimum stem length, and a loop size is greater than a minimum loop size.

26. The method of claim 25, wherein the initial quality score is approximately zero, the match number is from about 0.5 to about 3.0, the partial match number is from about 0.25 to about 1.5, five prime bulge number is from about −0.5 to about −6.0, the three prime bulge number is from about −0.5 to about −6.0, and the mismatch number is from about −0.5 to about −6.0.

27. The method of claim 25, wherein the threshold value is from about 5 to about 15, the minimum stem length is from about 5 to about 25 base pairs, and the minimum loop size is from about 3 to about 10 bases.

28. The method of claim 23, wherein the open-end method comprises:
(a) creating a two-dimensional dynamic programming table for each window to fit the sequential bases of each window along a horizontal top of the two-dimensional dynamic programming table and to fit the sequential bases of each window along a vertical left side of the two-dimensional dynamic programming table;
(b) representing the sequential bases of each window along the horizontal top of the two-dimensional dynamic programming table, forming a horizontal base top;
(c) representing the sequential bases of each window from the opposite direction along the vertical left side starting at the horizontal top of the two-dimensional dynamic programming table, forming a vertical base side;
(d) calculating a table quality score for entry into each cell of the two-dimensional dynamic programming table corresponding to each base-base interaction between the horizontal base top and the vertical base side using a scoring method, comprising (i) adding a match number to an initial quality score for each A-U, U-A, C-G, or G-C base match, forming a cumulative score, (ii) adding a partial-match number to the cumulative score for each G-U or U-G base match, (iii) adding a five-bulge number to the cumulative score for each 5 prime side bulge, (iv) adding a three-bulge number to the cumulative score for each 3 prime side bulge, and (v) adding a mismatch number to the cumulative score for each A-A, C-C, G-G, U-U, A-C, C-A, A-G, G-A, C-U, or U-C mismatch;
(e) locating a highest value of each table quality score corresponding to the optimum base pairing for each window; and
(f) storing the highest value and corresponding base sequence of each window when the highest value exceeds a threshold value, a stem length exceeds a minimum stem length, and a loop size is greater than a minimum loop size.

29. The method of claim 28, wherein the initial quality score is approximately zero, the match number is from about 0.5 to about 3.0, the partial match number is from about 0.25 to about 1.5, five prime bulge number is from about −0.5 to about −6.0, the three prime bulge number is from about −0.5 to about −6.0, and the mismatch number is from about −0.5 to about −6.0.

30. The method of claim 28, wherein the threshold value is from about 5 to about 15, the minimum stem length is from about 5 to about 25 base pairs, and the minimum loop size is from about 3 to about 10 bases.

31. The method of claim 18, further comprising:
ranking the interfering stem-loop sequences obtained from the dynamic programming method according to stem-loop quality, heterogeneity, and conservation;
selecting the interfering stem-loop sequences having a high ranking;
screening the interfering stem-loop sequences having a high ranking by complimentary pairing to a gene sequence of the target organism using a pairing method; and
selecting the interfering stem-loop sequences having a complimentary pairing to a gene sequence of the target organism using a parsing method.

32. The method of claim 31, wherein measurement of the heterogeneity comprises:
(a) measuring contiguous dinucleotide repeats; and
(b) rejecting the stem-loop structures having about 2 to about 15 dinucleotide repeats.

33. The method of claim 31, wherein measurement of the conservation comprises:
(a) measuring repeats of stem-loop structures located in the candidate genome; and
(b) rejecting the stem-loop structures having approximately zero repeats.

34. The method of claim 31, wherein the BLAST method comprises:
(a) preparing a stem-loop structures data file for submission by formatting the stem-loop structures data file;
(b) running the stem-loop structures data file; and
(c) retrieving and storing a BLAST output data file.

35. The method of claim 31, wherein the parsing method comprises the steps of:
(a) reading the BLAST output data file from a computer readable medium;
(b) parsing the BLAST output data file; and
(c) storing base sequence data when a base sequence of a candidate stem-loop structure has a base match of about 5 to about 50 matches to a candidate genome.

36. The method of claim 31, further comprising:
synthesizing the interfering stem-loop sequences having a complimentary pairing using a phosphoramidite chemistry method;
transfecting cells taken from the target organism with the interfering stem-loop sequences having a complimentary pairing to form transfected target cells using an assay method; and
identifying the transfected target cells that display a target phenotype.

37. The method of claim 36, wherein the phosphoramidite chemistry method comprises:
synthesizing a stem-loop structure using a Pol III RNA polymerase promoter on a chip array.

38. The method of claim 36, wherein the assay method is a transcription factor reporter assay.

39. The method of claim 36, wherein the target phenotype is cell survival after programmed cell death.

40. The method of claim 18, wherein the candidate genome comprises at least two strains of a viral genome.

41. The method of claim 41, wherein the viral genome is a pox viral genome.

42. The method of claim 18, wherein the candidate genome comprises a sequenced genome.

43. The method of claim 18, wherein the viral genome is a sequence obtained from a viral family, wherein the viral family is selected from the group consisting of:
“CrPV-like viruses”, “HEV-like viruses”, “SNDV-like viruses”, Adenoviridae, Allexivirus, Arenaviridae, Arteriviridae, Ascoviridae, Asfarviridae, Astroviridae, Baculoviridae, Barnaviridae, Benyvirus, Bimaviridae, Bomaviridae, Bromoviridae, Bunyaviridae, Caliciviridae, Capillovirus, Carlavirus, Caulimoviridae, Circoviridae, Closteroviridae, Comoviridae, Coronaviridae, Corticoviridae, Cystoviridae, Deltavirus, Filoviridae, Flaviviridae, Foveavirus, Furovirus, Fuselloviridae, Geminiviridae, Hepadnaviridae, Herpesviridae, Hordeivirus, Hypoviridae, Idaeovirus, Inoviridae, Iridoviridae, Leviviridae, Lipothrixviridae, Luteoviridae, Marafivirus, Metaviridae, Microviridae, Myoviridae, Nanovirus, Namaviridae, Nodaviridae, Ophiovirus, Orthomyxoviridae, Ourmiavirus, Papillomaviridae, Paramyxoviridae, Partitiviridae, Parvoviridae, Pecluvirus, Phycodnaviridae, Picornaviridae, Plasmaviridae, Podoviridae, Polydnaviridae, Polyomaviridae, Pomovirus, Potexvirus, Potyviridae, Poxviridae, Pseudoviridae, Reoviridae, Retroviridae, Rhabdoviridae, Rhizidiovirus, Rudiviridae, Sequiviridae, Siphoviridae, Sobemovirus, Tectiviridae, Tenuivirus, Tetraviridae, Tobamovirus, Tobravirus, Togaviridae, Tombusviridae, Totiviridae, Trichovirus, Tymovirus, Umbravirus, Varicosavirus, and Vitivirus.

44. An RNAi composition, for treating a condition in a target organism, comprising:
a composition composed of at least one type of stem-loop structure selected from the group consisting of SEQ ID NOs. 1-52 and combinations thereof.

45. The RNAi composition of claim 44, wherein the composition is composed of at least one type of stem-loop structure selected from the group consisting of SEQ ID NOs. 1, 2, 3, 6, 9, 10, and 11 and combinations thereof.

46. A pharmaceutical composition, for treating a condition in a target organism, comprising:
a composition composed of at least one type of stem-loop structure selected from the group consisting of SEQ ID NOs. 1-52 and combinations thereof and a pharmaceutically acceptable carrier.

47. The pharmaceutical composition of claim 46, wherein the composition is composed of at least one type of stem-loop structure selected from the group consisting of SEQ ID NOs. 1, 2, 3, 6, 9, 10, and 11 and combinations thereof and a pharmaceutically acceptable carrier.

48. A method for treatment of a condition in a target organism, comprising:
administering an effective amount of a composition composed of at least one type of stem-loop structure selected from the group consisting of SEQ ID NOs. 1-52 and combinations thereof.

49. The method of claim 48, wherein the method comprises administering an effective amount of the composition composed of at least one type of stem-loop structure selected from the group consisting of SEQ ID NOs. 1, 2, 3, 6, 9, 10, and 11 and combinations thereof.

50. An RNAi composition, for treating a condition in a target organism, comprising:
a stem-loop structure and combinations thereof obtained by the method according to claim 18.

51. An RNAi composition, for treating a condition in a target organism, comprising:
a stem-loop structure and combinations thereof obtained by the method according to claim 31.

52. An RNAi composition, for treating a condition in a target organism, comprising:
a stem-loop structure and combinations thereof obtained by the method according to claim 32.

53. A pharmaceutical composition, for treating a condition in a target organism, comprising:
a pharmaceutically acceptable carrier and a stem-loop structure and combinations thereof obtained by the method according to claim 18.

54. A pharmaceutical composition, for treating a condition in a target organism, comprising:
a pharmaceutically acceptable carrier and a stem-loop structure and combinations thereof obtained by the method according to claim 31.

55. A pharmaceutical composition, for treating a condition in a target organism, comprising:
a pharmaceutically acceptable carrier and a stem-loop structure and combinations thereof obtained by the method according to claim 32.


SEQUENCE LISTING (52 sequences)

SEQ ID NO: 1 - DNA, 21 bp, Pox virus
  Nucleic Acid
tatttatgtattatttattta

SEQ ID NO: 2 - DNA, 22 bp, Pox virus
  Nucleic Acid
tatttttatttgataatctata

SEQ ID NO: 3 - DNA, 22 bp, Pox virus
  Nucleic Acid
atttttataatattttgtaata

SEQ ID NO: 4 - DNA, 20 bp, Pox virus
  Nucleic Acid
aaaataatttattacattta

SEQ ID NO: 5 - DNA, 24 bp, Pox virus
  Nucleic Acid
tttaaaaaaagaaaaaaaaaggca

SEQ ID NO: 6 - DNA, 20 bp, Pox virus
  Nucleic Acid
tttgtattaaaatatataga

SEQ ID NO: 7 - DNA, 24 bp, Pox virus
  Nucleic Acid
atatttaattgataattataatat

SEQ ID NO: 8 - DNA, 22 bp, Pox virus
  Nucleic Acid
ttttaataaaacaaaagttgtt

SEQ ID NO: 9 - DNA, 21 bp, Pox virus
  Nucleic Acid
gaagaagaaaagaaaaaatat

SEQ ID NO: 10 - DNA, 22 bp, Pox virus
  Nucleic Acid
atttaaaaaattcatttttaat

SEQ ID NO: 11 - DNA, 23 bp, Pox virus
  Nucleic Acid
aaaaataatttaaaaataattaa

SEQ ID NO: 12 - DNA, 23 bp, Pox virus
  Nucleic Acid
aatataaataataaaacaatatt

SEQ ID NO: 13 - DNA, 27 bp, Pox virus
  Nucleic Acid
gttataaaatgacaatatatttttaaa

SEQ ID NO: 14 - DNA, 65 bp, Pox virus
  Nucleic Acid
tatttatgtattatttatttaccaaaagaaatagatcttacttattaagcagatatgcataaata

SEQ ID NO: 15 - DNA, 43 bp, Pox virus
  Nucleic Acid
atatttttatttgataatctatagatgtatcaaataacgatat

SEQ ID NO: 16 - DNA, 81 bp, Pox virus
  Nucleic Acid
aatttttataatattttgtaatataatataaaacattatcttggttaatatcaatgttatttataaaatttaatacagatt

SEQ ID NO: 17 - DNA, 70 bp, Pox virus
  Nucleic Acid
tgtaaaataatttattacatttatattactttgaacaacaataggataaaaatatgataataattttaca

SEQ ID NO: 18 - DNA, 64 bp, Pox virus
  Nucleic Acid
tgtttttaaaaaaagaaaaaaaaaggcattattttttctttttttttctttttttttaaaaata

SEQ ID NO: 19 - DNA, 79 bp, Pox virus
  Nucleic Acid
gaataattaattatattatacatattaatatttgtattaaaatatatagataatatatgtattattaatgcaattattc

SEQ ID NO: 20 - DNA, 96 bp, Pox virus
  Nucleic Acid
tgtattggtatatttaattgataattataatattgaaatatttaacatgtttttgctaatttatgcttattctcattatctaaatatgttaatata

SEQ ID NO: 21 - DNA, 108 bp, Pox virus
  Nucleic Acid
tttattttttttaaaaccgaaattatcttggcttttaataaaacaaaagttgttaataatccaatcgacaaatttaaagatacatttttgttttgcaaagaaaataaa

SEQ ID NO: 22 - DNA, 82 bp, Pox virus
  Nucleic Acid
atgataggatgtatttctgctaattcaaatttaacagaagaagaaaagaaaaaatataaaagtgaaggatatattttattat

SEQ ID NO: 23 - DNA, 96 bp, Pox virus
  Nucleic Acid
atgtactattttatcaaaatgatactactgatattaaagatttaaaaaattcatttttaatgattgatactagtattgtagataaatattgtatat

SEQ ID NO: 24 - DNA, 119 bp, Pox virus
  Nucleic Acid
tattgatattgataattcgtattatagaggaaatgcacttattgagttcctagataaaaataatttaaaaataattaatgatataattttatctaataatactaataattggattgatg

SEQ ID NO: 25 - DNA, 98 bp, Pox virus
  Nucleic Acid
tttaatatatccatatttattttttattatataccttttcaaaaaaaagatattttaatataaataataaaacaatattataaatggatattattgaa

SEQ ID NO: 26 - DNA, 113 bp, Pox virus
  Nucleic Acid
atttaaatatttattataatagttatgatgtattgaattttttttttcatagaataaataaatcatattaattaaatttatatatgttataaaatgacaatatatttttaaat

SEQ ID NO: 27 - DNA, 64 bp, Pox virus
  Nucleic Acid
catgcaagatccgtatttatgtattatttatttaccaaaagaaatagatcttacttattaagca

SEQ ID NO: 28 - DNA, 53 bp, Pox virus
  Nucleic Acid
catgcaagatccgatatttttatttgataatctatagatgtatcaaataacga

SEQ ID NO: 29 - DNA, 72 bp, Pox virus
  Nucleic Acid
catgcaagatccgaatttttataatattttgtaatataatataaaacattatcttggttaatatcaatgtta

SEQ ID NO: 30 - DNA, 67 bp, Pox virus
  Nucleic Acid
catgcaagatccgtgtaaaataatttattacatttatattactttgaacaacaataggataaaaata

SEQ ID NO: 31 - DNA, 70 bp, Pox virus
  Nucleic Acid
catgcaagatccgtgtttttaaaaaaagaaaaaaaaaggcattattttttctttttttttcttttttttt

SEQ ID NO: 32 - DNA, 72 bp, Pox virus
  Nucleic Acid
catgcaagatccggaataattaattatattatacatattaatatttgtattaaaatatatagataatatatg

SEQ ID NO: 33 - DNA, 80 bp, Pox virus
  Nucleic Acid
catgcaagatccgtgtattggtatatttaattgataattataatattgaaatatttaacatgtttttgctaatttatgct

SEQ ID NO: 34 - DNA, 80 bp, Pox virus
  Nucleic Acid
gcaagatccgtttattttttttaaaaccgaaattatcttggcttttaataaaacaaaagttgttaataatccaatcgaca

SEQ ID NO: 35 - DNA, 73 bp, Pox virus
  Nucleic Acid
catgcaagatccgatgataggatgtatttctgctaattcaaatttaacagaagaagaaaagaaaaaatataaa

SEQ ID NO: 36 - DNA, 80 bp, Pox virus
  Nucleic Acid
catgcaagatccgatgtactattttatcaaaatgatactactgatattaaagatttaaaaaattcatttttaatgattga

SEQ ID NO: 37 - DNA, 80 bp, Pox virus
  Nucleic Acid
caagatccgtattgatattgataattcgtattatagaggaaatgcacttattgagttcctagataaaaataatttaaaaa

SEQ ID NO: 38 - DNA, 78 bp, Pox virus
  Nucleic Acid
gcaagatccgtttaatatatccatatttattttttattatataccttttcaaaaaaaagatattttaatataaataat

SEQ ID NO: 39 - DNA, 80 bp, Pox virus
  Nucleic Acid
caagatccgatttaaatatttattataatagttatgatgtattgaattttttttttcatagaataaataaatcatattaa

SEQ ID NO: 40 - DNA, 55 bp, Pox virus
  Nucleic Acid
tgtactgtgaattcaaaaaatatttatgcatatctgcttaataagtaagatctat

SEQ ID NO: 41 - DNA, 44 bp, Pox virus
  Nucleic Acid
tgtactgtgaattcaaaaaaatatcgttatttgatacatctata

SEQ ID NO: 42 - DNA, 63 bp, Pox virus
  Nucleic Acid
tgtactgtgaattcaaaaaaaatctgtattaaattttataaataacattgatattaaccaaga

SEQ ID NO: 43 - DNA, 57 bp, Pox virus
  Nucleic Acid
tgtactgtgaattcaaaaaatgtaaaattattatcatatttttatcctattgttgtt

SEQ ID NO: 44 - DNA, 61 bp, Pox virus
  Nucleic Acid
tgtactgtgaattcaaaaaatatttttaaaaaaaaagaaaaaaaaagaaaaaataatgcct

SEQ ID NO: 45 - DNA, 62 bp, Pox virus
  Nucleic Acid
tgtactgtgaattcaaaaaagaataattgcattaataatacatatattatctatatatttta

SEQ ID NO: 46 - DNA, 71 bp, Pox virus
  Nucleic Acid
tgtactgtgaattcaaaaaatatattaacatatttagataatgagaataagcataaattagcaaaaacatg

SEQ ID NO: 47 - DNA, 76 bp, Pox virus
  Nucleic Acid
tgtactgtgaattcaaaaaatttattttctttgcaaaacaaaaatgtatctttaaatttgtcgattggattattaa

SEQ ID NO: 48 - DNA, 63 bp, Pox virus
  Nucleic Acid
tgtactgtgaattcaaaaaaataataaaatatatccttcacttttatattttttcttttcttc

SEQ ID NO: 49 - DNA, 69 bp, Pox virus
  Nucleic Acid
gtactgtgaattcaaaaaaatatacaatatttatctacaatactagtatcaatcattaaaaatgaattt

SEQ ID NO: 50 - DNA, 79 bp, Pox virus
  Nucleic Acid
tgtactgtgaattaaaaaacatcaatccaattattagtattattagataaaattatatcattaattatttttaaattat

SEQ ID NO: 51 - DNA, 70 bp, Pox virus
  Nucleic Acid
gtactgtgaattcaaaaaattcaataatatccatttataatattgttttattatttatattaaaatatct

SEQ ID NO: 52 - DNA, 79 bp, Pox virus
  Nucleic Acid
tgtactgtgaattcaaaaaaatttaaaaatatattgtcattttataacatatataaatttaattaatatgatttattta